(19) United States
(12) Patent Application Publication
(10) Pub. No.: US 2006/0251620 A1
(43) Pub. Date: Nov. 9, 2006

(54) Inducible alphaviral/orip based gene expression system

(76) Inventors: Lidia Ivanova, Zurich-Schlieren (CH); Wolfgang A. Renner, Kilchberg (CH); Philippe Saudan, Pfungen (CH)

Correspondence Address: NATH & ASSOCIATES, 112 South West Street, Alexandria, VA, 22314, US

(21) Appl. No.: 10/524,818

(22) PCT Filed: Aug. 21, 2003
(86) PCT No.: PCT/EP03/09291

§ 371 Date: Dec. 5, 2005

Related U.S. Application Data
(60) Provisional application No. 60/404,928, filed on Aug. 22, 2002.
(60) Provisional application No. 60/432,259, filed on Dec. 11, 2002.

(51) Int. Cl. A61K 48/00; C12N 15/86; C07H 21/04

(52) U.S. Cl. 424/93.2; 435/456; 536/23.2

(57) ABSTRACT

The present invention relates to compositions and methods that allow the production of polypeptides and untranslated RNA molecules in host cells. More specifically, the invention provides nucleic acid molecules, expression systems, recombinant host cells, methods and kits, which enable the production and/or isolation and/or purification of polypeptides and untranslated RNA molecules. The compositions and methods of the invention can be applied in a regulatable expression system that is transiently transfected into mammalian host cells.

### BACKGROUND OF THE INVENTION

1. Field of the Invention

The present invention relates to compositions and methods for the production of polypeptides and/or untranslated RNA molecules in host cells. The invention provides nucleic acid molecules, expression systems, host cells, methods and kits that are useful for the production of polypeptides and/or untranslated RNA molecules.

2. Related Art

Process development for biopharmaceuticals is influenced by product quality and economy of the manufacturing process. The economic production of recombinant proteins in mammalian cells is dependent on the selection of the producing cell lines. The classical approach is the use of stable expression systems. These systems are based on chromosomal integration of an expression plasmid into the genome of the host cell.

An alternative to stable expression systems is transient gene expression. For example, transient gene expression in, e.g., mammalian cells, at reactor scale is becoming increasingly important for the rapid production of recombinant proteins. Large scale applications of transient expression systems for recombinant protein production have been reported using COS, HEK 293 and BHK21 cells (Blasey et al., 1996, Cytotechnology, 18, 138-192; Jordan et al., 1998, Cytotechnology, 26, 39-47; Wurn and Bernard, 1999, Curr Opin Biotechn 10, 156-159; Meissner et al., 2001, Biotechn and Bioeng, 75, 2, 197-203).

Transient expression can be performed by infection (using, e.g., viral vectors) or by transfection of the appropriate vector. Transfection can be performed with either a non-replicating or a replicating DNA vector or with a virus derived RNA vector.

A variety of virus vectors for expression in higher eukaryotic cells have been developed by using recombinant DNA technology and introducing genes of interest into virus genomes. The initial viruses used in vector development were DNA viruses such as Adeno-associated viruses or Poxviruses as well as RNA viruses replicating through DNA intermediates such as Retroviruses.

Episomal replicating vectors provide certain advantages over classical expression systems. Some DNA viruses, such as Adeno-, Papilloma-, Polyoma-, Hepadna-, and Herpesviruses, do not normally integrate into the host genome, but replicate episomally (extrachromosomally) in the nucleus of a host cells such as in a mammalian host cell. This process may involve both virus trans-acting factors and the host cell replication machinery. Episomal replicating vectors derived from these viruses generally contain a replication origin and at least one viral protein, e.g., an initiator protein. Examples of such initiator proteins are large T-antigen for SV40, E1/E2 for BPV, and EBNA-1 for EBV.

Some host factors are able to recognize viral origins of replication and initiate replication. For example human S/MAR (scaffold/matrix attached region from the human interferon-beta gene) is able to maintain the episomal replication of a vector containing SV 40 origin of replication. On the other hand certain viral origins of replication (ORI) are recognized by corresponding specific virally-encoded proteins. For example, SV40 or polyoma virus ORI is recognized by large T antigen, BPV (Bovine papilloma virus) ORI is recognized by E1/E2 complex, EBV (Epstein-Barr virus) ORI is recognized by EBNA1 protein, and yeast origin of replication (ARS element) is recognized by ORE (origin recognition element) replication (Bode et al., 2001 Gene Ther Mol Biol, v6, 3346).

The double strand DNA genome of Epstein-Barr virus EBV (165 kb) is maintained in the latently infected cells as a large circular episome. Amplification of this episome is mediated by a cis-acting sequence such as the origin of replication (OriP), and one of the viral proteins, the Epstein-Barr viral nuclear antigen 1 (EBNA1). In a minimal system the presence of both OriP (1.8 kb region) and EBNA-1 is sufficient for replication and stabilization of recombinant plasmids. The exact function of EBNA-1 is still not clear, although the protein has a DNA binding domain. Generally EBNA-1 is involved in plasmid maintenance after completion of the synthesis and in plasmid segregation by dividing cells during proliferation. The OriP region is recognized also directly by the cellular DNA synthesizing machinery and is replicated during the cell cycle.

Other gene expression systems are based on alphaviruses (Lundstrom, K., Curr. Opin. Biotechnol. 8:578-582 (1997)). Several members of the alphavirus family, e.g., Sindbis virus (Xiong, C. et al., Science 243:1188-1191 (1989); Schlesinger, S., Trends Biotechnol. 11:18-22 (1993)), SFV (Liljeström P. & Garoff, H., Bio/Technology 9:1356-1361 (1991)) and others (Davis, N. L. et al., Virology 171:189-204 (1989)) have received considerable attention for the use as virus-based expression vectors for a variety of different proteins (Lundstrom, K., Curr. Opin. Biotechnol. 8:578-582 (1997); Liljeström, P., Curr. Opin. Biotechnol. 5:495-500 (1994)).

Alphaviruses are positive stranded RNA viruses which replicate their genomic RNA entirely in the cytoplasm of the infected cell and without a DNA intermediate (Strauss, J. and Strauss, E., Microbiol. Rev. 58:491-562 (1994)). The concept that alphaviruses can be developed as expression vectors was first established nearly ten years ago (Xiong, C. et al., Science 243:1188-1191 (1989)). Since then, several improvements have made the use of these RNA replicons as expression vectors more practical (Lundstrom, K., Curr. Opin. Biotechnol. 8:578-582 (1997)).

DNA vectors have been developed for both Sindbis virus (Herweijer, H. et al., Hum. Gene Ther. 6:1495-1501 (1995); Dubensky, T. W. et al., J. Virol. 70:508-519 (1996)) and SFV (Berglund, P. et al., Trends Biotechnol. 14:130-134 (1996)). Eukaryotic promoters are introduced in these vectors upstream from the alphavirus replicase gene (consisting of the four non-structural protein genes (nsP1-4)), which are translated as one or two polyproteins which are then proteolytically cleaved. DNA is transcribed into RNA from the recombinant eukaryotic promoter in the nucleus and transported to the cytoplasm, where the replicase catalyzes the replication of the alphavirus RNA molecule as during normal replication of the alphavirus RNA molecule (Strauss, J. and Strauss, E., Microbiol. Rev. 58:491-562 (1994)). Only transient expression of heterologous sequences has been possible until recently due to the cytopathogenicity of the alphavirus replicase (Lundstrom, K., Curr. Opin. Biotechnol. 8:578-582 (1997)).

Among the features which make Alphavirus vectors attractive as transient expression systems are the easy construction of plasmid clones, a rapid gene expression (within 24 h), a high level of expression, a wide host range of the virus (many insect, avian and mammalian cells), production of high-titered stocks of infectious particles and low risk of cell transformation due to the RNA nature of the genome and its replication entirely in the host cell cytoplasm without going through DNA intermediates. (Schlesinger S., 2000, Exp. Opin. Biol. Ther., 1, 2, 177-191; Schlesinger S., Adv. Virus res., v55, 565-577; Garoff, H. and K. J. Li, 1998, Curr. Opin. Biotechnol., 9, 464-469; Lundstrom, K., 1997, Curr. Opin. Biotechnol., 8, 578-583; Xiong et al., 1989, Science, 243, 1188-1191). Blasey et al. compared the Semliki Forest Virus (SFV) expression system with the Epstein-Barr Virus (EBV) system (pCEP4 and HEK293EBNA cells) for expression of 5-HT3 receptors and found that both systems allow the production of functional recombinant protein within days to weeks at very high comparable levels (in: Animal Cell Technology, 1999, pp 331-337).

One of the variants of alphavirus vectors is the replicon, which is capable of self-replication in the host cytoplasm after introduction into the cultured cells by transfection. In this replicon, viral structural protein genes have been replaced by a gene of interest (GOI). Such a replicon can be introduced into cells as RNA or cDNA if placed under the control of an eukaryotic promoter or a promoter, which is recognized by the host cell machinery. (Agapov et al., Proc. Natl. Acad. Sci. USA 95:12989-12994 (1998)).

RNA viruses have been engineered into chimeric viruses producing heterologous proteins. Such RNA virus vectors have been developed from Alphaviruses (with positive strand genome), Influenza and VSV (with negative strand genome). One of the major advantages of these viruses is their RNA-only replication strategy performed entirely in the cell cytoplasm and eliminating the risk of genome integration.

Adeno-, Adeno-associated and Retrovirus vectors can be used as gene therapy vectors and/or as expression vectors, especially for large-scale protein production. Herpesvirus derivative vectors containing a replication origin have been used for bioreactor protein production after transient transfection of a suitable cell line carrying a replication initiation gene. (Durocher et al., Nucleic Acids Res. 30(2):E9 (2002); Meissner et al., Biotechnol. Bioeng. 75(2):197-203 (2001)). In these experiments, 293E cells (293 human embryo kidney cells expressing Epstein-Barr virus nuclear antigene—EBNA-1) were used which provided a replication initiation factor in trans to a plasmid carrying EBV origin of replication—OriP. Reporter genes (encoding secreted, intracellular or transmembrane proteins) were constitutively expressed over a period of several days.

Hybrid vectors have also been reported, mainly as gene delivery systems or for use in virus biology research, such as combinations between EBV episomes and Herpes-, Adeno-(DNA) or Retro-(RNA-DNA) viruses (Sena-Esteves M. et al, 1999, Virol. December; 73(12):10426-39; Tan B T. et al., 1999, J Virol. September, 73(9):7582-9; Grignani F. et al., 1998, Cancer Res January, 1; 58(1):14-9; Wang S. and Vos J M., 1996, J. Virol. December, 70(12):8422-30). EBV episomes are nuclear plasmids that are stably maintained through multiple cell divisions in primate and canine cells (J. L. Yates, N. Warren, and B. Sugden, 1985, Nature 313:812-815).

The ability to precisely control the expression of genes introduced into animal or human cells, or in whole organisms, enables significant progress in many areas of biology and medicine. For instance, methods that allow the intentional manipulation of gene expression will facilitate the analysis of genes whose expression cannot be tolerated constitutively.

To be of broad benefit, gene regulation techniques preferably allow for rapid, robust, precise and reversible induction of gene activity. As reviewed in Saez, E. et al., (Curr. Opin. Biotechnol. 8:608-616 (1997)), an ideal system would fulfill the following requirements:

1. Specificity—The system should be indifferent to endogenous factors and activated only by exogenous stimuli.

2. Non-interference—The components of the system should not affect unintended cellular pathways.

3. Inducibility—In the inactive state, the basal activity of the system should be minimal, while in the active state high levels of gene expression should be rapidly inducible.

4. Bioavailability of the inducer—Inducing stimuli should rapidly penetrate to the site of interest.

5. Reversibility—Inducing stimuli should clear swiftly to allow the system to rapidly return to the inactive state.

One common system currently in use for the regulation of gene expression is the tetracycline-based system (Gossen and Bujard, Proc. Natl. Acad Sci. USA 89:5547 (1992)). This system is based on the continuous expression of a fusion protein with the tetracycline repressor protein (tetR) being converted into an activator by fusion to the transcriptional activation domain of the VP16 protein. The tTA system is useful for, e.g., inducible gene expression and has been successfully used for the production of a number of proteins (Wimmel et al., Oncogene 9:995 (1994); Früh et al., EMBO J. 13:3236 (1994); Yu et al., J. Virol. 70:4530 (1996)). However, there are certain disadvantages associated with the tTA system. (Schocket et al., Proc. Natl. Acad. Sci. USA 92:6522 (1995); Howe et al., J. Biol. Chem. 23:14168 (1995); Schocket and Schatz, Proc. Natl. Acad. Sci. USA 93:5173 (1996); Bohl et al., Nat. Med. 3:299 (1997); Furth et al., Proc. Natl. Acad. Sci. USA 91:9302 (1994); Hennighausen et al., J. Cell. Biochem. 59:463 (1995); Kistner et al., Proc. Natl. Acad Sci. USA 93:10933 (1996); Hoffmann et al., Nucleic Acids Res. 25:1078 (1997)).

Inducible expression systems based on viral expression vectors (e.g., aphaviral vectors) are described in WO 99/50432 (incorporated herein by reference in its entirety). An exemplary alphaviral vector is pCytTs (also known as CytTs; cf. WO 99/50432). Using these systems, it is possible to precisely induce and regulate gene expression. This precise regulation results from the use of a temperature-sensitive RNA-dependent RNA polymerase (i.e., a replicase), which only replicates RNA molecules, to form new RNA molecules, at permissive temperatures (Boorsma et al. Nature Biotech. 18:429-432 (2001)). Due to the extremely tight regulation of these systems it is possible to obtain regulated gene expression even under conditions of transient transfection allowing the production of toxic proteins under these conditions.

Another example of an alphaviral expression vector is described in Lundstrom et al., FEBS Lett 504:99-103 (2001). Lundstrom describe a rapidly generated high-titer Semliki Forest virus (SFV) vectors capable of infecting numerous mammalian cell lines and primary cell cultures, resulting in high levels of transgene expression. SFV-based expression of transmembrane receptors has been characterized by specific ligand-binding activity and functional responses. Adaptation of the SFV technology for mammalian suspension cultures has allowed the production of hundreds of milligrams of recombinant receptor for purification and structural studies. The same SFV stock solutions used for the infection of mammalian cells in culture have also been successfully applied for efficient transgene expression in organotypic hippocampal slices, as well as in vivo in rodent brain.

A need exists in the art, however, for compositions and methods that allow the production of polypeptides and untranslated RNA molecules.

### BRIEF SUMMARY OF THE INVENTION

The present invention provides compositions and methods that allow the production of polypeptides and/or untranslated RNA molecules. More specifically, the invention provides nucleic acid molecules, expression systems, recombinant host cells that permit the production of polypeptides and/or untranslated RNA molecules. Also provided are methods for making the nucleic acid molecules, expression systems and host cells of the invention. The invention further provides methods for producing polypeptides and/or untranslated RNA molecules. The invention also provides kits that comprise the nucleic acid molecules, expression systems and/or recombinant host cells of the invention.

In certain embodiments, the invention provides compositions and methods for regulated, expression of polypeptides or untranslated RNA molecules in recombinant host cells, preferably by way of transfection. The present invention also provides nucleic acid molecules and methods which allow, e.g., rapid and high level production of specific RNA molecules produced in, e.g., transiently transfected, recombinant host cells.

According to certain aspects of the invention, novel, preferably inducible, potent and rapid expression systems are provided comprising one or more nucleic acid molecules. The nucleic acid molecules of the expression systems of the invention may be maintained at, or accumulated to, multiple copies in the nuclei of recombinant host cells, preferably of transiently transfected mammalian cells. In certain embodiments, RNA self replication may lead to the accumulation of a high number of RNA molecules in the cytoplasm of the transfected cells and subsequent translation of the polypeptide of interest. The nucleic acid molecules and expression systems of the invention are particularly useful for transient transfection.

The present invention further provides nucleic acid molecules and methods which allow inducible rapid and high level production of specific RNA molecules produced in, preferably transiently transfected, recombinant host cells. In certain embodiments, RNA self-replication is preferably inducible and triggered by a temperature shift. Thus, the high production levels, in certain embodiments, are achieved by the use of a temperature-sensitive RNA-dependent RNA polymerase (i.e., a replicase) which only replicates RNA molecules to form new RNA molecules at permissive temperatures. Moreover, if desired, the amount of RNA or polypeptide can be tightly regulated by the use of a temperature-sensitive RNA-dependent RNA polymerase. This facilitates, in particular, production of growth-inhibitory or toxic polypeptides or RNA-species.

In a general aspect, the present invention provides for a nucleic acid molecule comprising (A) a first polynucleotide element which encodes an RNA molecule, said RNA molecule comprising (a) at least one cis-acting sequence element; (b) a first nucleotide sequence comprising a first open reading frame, said first open reading frame having a nucleotide sequence encoding an RNA-dependent RNA polymerase; and (c) at least one second nucleotide sequence selected from the group consisting of (i) a second open reading frame encoding a polypeptide; (ii) a nucleotide sequence complementary to all or part of the second open reading frame of (i); and (iii) a nucleotide sequence encoding an untranslated RNA molecule or complement thereof; wherein said second nucleotide sequence is operably linked to a promoter which is recognized by said RNA-dependent RNA polymerase; (B) a second DNA element comprising an origin of replication; and (C) a third DNA element encoding a replication initiation factor capable of recognizing the origin of replication. Thus, the replication initiation factor preferably recognizes the origin of replication leading to replication of the second polynucleotide and second DNA element, respectively, comprising the origin of replication.

In certain embodiments, the RNA-dependent RNA polymerase is, e.g., a non-cytopathic RNA-dependent RNA polymerase. In other embodiments, the RNA-dependent RNA polymerase is, e.g., a temperature-sensitive RNA-dependent RNA polymerase. In yet other embodiments, the RNA-dependent RNA polymerase is, e.g., a non-cytopathic, temperature-sensitive RNA-dependent RNA polymerase.

The nucleic acid molecules of the invention, in certain embodiments, comprise a 5′ promoter which is capable of initiating transcription in vivo, 5′ and/or 3′ sequences enabling replication of the RNA molecule (cis-acting sequence elements), and a subgenomic promoter 5′ to the gene of interest, as well as a sequence of interest which is translatable only after one or more RNA-dependent RNA replication events. These RNA-dependent RNA replication events are catalyzed by a, preferably regulatable, RNA-dependent RNA polymerase which may be encoded by the same mRNA molecule that is produced by transcription of the DNA vector or by a different mRNA molecule.

The inventive nucleic acid molecules, expression systems and vector systems of the present invention thus allow for large-scale transient transfection and very rapid production of polypeptides eliminating the need of isolating stably transformed high producing cell clones. The preferred embodiment and hereby the inducibility of the vector makes the invention especially suitable for the production of cytotoxic polypeptides, e.g., polypeptides that are detrimental to the viability of a host cell.

In a preferred embodiment of the present invention, the origin of replication and the replication initiation factor are derived from a DNA virus. For example, the origin of replication may be derived from a herpes virus and the replication initiation factor may be the Epstein-Barr virus nuclear antigen 1 (EBNA1). However, replication initiation factors may also derive from other viruses or be cellular factors, which are capable of recognizing or recognize viral origins of replication. As indicated, in a further embodiment, the RNA-dependent RNA-polymerase is temperature-sensitive and temperature-regulated, respectively. Preferably, the RNA-dependent RNA-polymerase is of viral, more preferably of alphaviral origin.

Thus, in a preferred embodiment, the present invention provides a hybrid vector and vector system combining the advantages of EBV episomal replication with the advantages of RNA virus replication, wherein the replication takes place entirely in the cytoplasm. This type of hybrid vector would be characterized by the maintenance of multiple copies of DNA due to polynucleotide replication, typically plasmid replication, followed by the accumulation of full-length transcripts containing the viral replicon. These viral full-length transcripts are then further exponentially amplified in the cytoplasm by the viral replicase leading to the accumulation of RNA virus replicons and protein synthesis. The system is extremely valuable for production of toxic polypeptides if the RNA virus replication is controllable or inducible.

In a specific embodiment, the invention includes the combination of a Herpesvirus mini-replicon unit, i.e. the cis-acting replication origin OriP (Origin of replication P) and the cis- or trans-acting gene product—EBNA-1 (Epstein-Barr virus nuclear antigen 1, a replication triggering factor) with the tightly regulated temperature inducible alphaviral expression system pCytTS, as disclosed in WO 99/504332. Due to the presence of OriP and EBNA-1 the introduced novel vector is maintained episomally (extrachromosomally) in the form of several DNA copies in the cell nuclei. These multiple DNA copies may be transcribed from a promoter, typically a CMV or RSV promoter, into CytTS RNA-replicons, but they remain inactive unless cells are being shifted to a certain temperature. Only after temperature induction replicon replication takes place, followed by RNA accumulation and translation in the cell cytoplasm and production of the polypeptide of interest.

In a further general aspect, the present invention provides a vector system comprising one or more nucleic acid molecules, wherein said one or more nucleic acid molecules comprise (A) a first polynucleotide which encodes an RNA molecule, said RNA molecule comprising (a) at least one cis-acting sequence element; (b) a first nucleotide sequence comprising a first open reading frame having a nucleotide sequence encoding an RNA-dependent RNA polymerase; and (c) at least one second nucleotide sequence selected from the group consisting of (i) a second open reading frame encoding a polypeptide; (ii) a sequence complementary to all or part of the second open reading frame of (i); and (iii) a sequence encoding an untranslated RNA molecule, or complement thereof; wherein said second nucleotide sequence is operably linked to a promoter which is recognized by said non-cytopathic RNA-dependent RNA polymerase; (B) a second polynucleotide comprising an origin of replication; and (C) a third polynucleotide encoding a replication initiation factor capable of recognizing the origin of replication. Thus, the replication initiation factor recognizes the origin of replication leading to replication of the second polynucleotide and second DNA element, respectively, comprising the origin of replication.

The invention further provides single- and multiple-vector systems for producing a polypeptide or untranslated RNA molecule. In a single-vector system, the first, second and third polynucleotide are present on the same nucleic acid molecule. In a multiple-vector system, the first, second and third polynucleotide elements are present on one or more separate nucleic acid molecules. In such a multiple vector system, at least one, preferably some or more preferably all vectors contain an origin of replication. Therefore, in preferred embodiments of the invention, the replication initiation factor and the origin of replication recognizing the replication initiation factor may be on the same nucleic acid molecule and plasmid, respectively, as it is the RNA-replicase or on a separate nucleic acid molecule and plasmid, respectively.

When sequences encoding the first and second open reading frame are present either on the same nucleic acid molecule or in the same vector (i.e., in a single-vector system), a region will preferably be present 5′ to the second open reading frame which inhibits translation of this open reading frame.

If a temperature sensitive RNA-dependent RNA polymerase (e.g., a temperature-sensitive replicase) is included within the compositions and methods of the invention, the temperature-sensitive replicase may be “cold” or “hot” sensitive and thus will only efficiently catalyze RNA-dependent RNA replication at temperatures either above or below the restrictive temperature. In one preferred embodiment, the compositions and methods of the invention comprise, or involve the use of, an RNA-dependent RNA polymerase that has replicase activity at temperatures below 34° C. which is at least five fold greater than the replicase activity exhibited at 34° C. or above. Preferably, the replicase activity at temperatures below 34° C. is at least ten fold greater than the replicase activity exhibited at 34° C. or above. In certain embodiments, the replicase activity at temperatures below 34° C. is at least one hundred fold greater than the replicase activity exhibited at 34° C. or above. Temperature-sensitive RNA-dependent RNA polymerases included within the present invention may, in certain embodiments, exhibit no (or undetectable) replicase activity at 34° C. or above.

In another aspect, the nucleic acid molecules and expression systems of the invention encode one or more cytokine, lymphokine, tumor necrosis factor, interferon, toxic protein, prodrug converting enzyme, or other polypeptide.

In yet another aspect, the nucleic acid molecules and expression systems of the invention encode an untranslated RNA molecule, such as an antisense RNA molecule, tRNA molecule, rRNA molecule, or ribozyme.

The invention also provides methods for making recombinant host cells comprising introducing nucleic acid molecules of the invention into host cells. Further provided are recombinant host cells produced by the introduction of nucleic acid molecules of the invention. In one embodiment, one, some or all of these recombinant host cells contain one or more nucleic acid molecules that comprise an RNA-dependent RNA polymerase and/or the replication initiation factor. The replication initiation factor, in certain embodiments, may be stably integrated into the genome of the one, some or all host cells.

The invention further provides isolated nucleic acid molecules comprising polynucleotides which comprise the nucleotide sequence of SEQ ID NO:21 (pCytTs-OriP) and SEQ ID NO:22 (pCytTs-OPE).

The present invention also provides methods for producing polypeptides and untranslated RNA molecules in recombinant host cells, said methods comprising introducing a nucleic acid molecule of the invention into a host cell to produce a recombinant host cell, and culturing the recombinant host cell under conditions suitable for expression of said polypeptide or untranslated RNA molecule. The methods of the invention may further comprise recovering said polypeptide or untranslated RNA molecule.

Methods are also provided for the expression of heterologous polypeptides, preferably the regulated expression of heterologous polypeptides, including cytokines, lymphokines, tumor necrosis factors, interferons, toxic polypeptides, and prodrug converting enzymes.

Further provided are polypeptides and untranslated RNA molecules produced by the methods of the invention.

The invention also provides methods for regulating the expression of a polypeptide or an untranslated RNA molecule, said method comprising introducing nucleic acid molecules of the invention into a host cell to produce a recombinant host cell and growing said recombinant host cell under suitable culture conditions. In certain embodiments, nucleic acid molecules encoding a temperature-sensitive RNA-dependent RNA-polymerase are used. When a temperature-sensitive RNA-dependent RNA-polymerase is used, the methods according to this aspect of the invention may further comprise changing the temperature of the recombinant host cell culture from either (i) a permissive temperature to a restrictive temperature, or (ii) a restrictive temperature to a permissive temperature.

In certain embodiments, the methods of the invention involve introducing the nucleic acid molecules of the invention into prokaryotic or eukaryotic host cells to produce a recombinant host cell, and then culturing said recombinant host cell under suitable culture conditions. The recombinant host cells may be cultured, e.g., in a serum-free or protein-free medium.

The present invention also provides pharmaceutical compositions comprising nucleic acid molecules of the invention and a pharmaceutically acceptable carrier.

Also included within the invention are kits comprising the nucleic acid molecules of the invention. Kits of the invention may additionally or alternatively comprise one or more expression systems of the invention and one or more recombinant host cells of the invention.

### BRIEF DESCRIPTION OF THE DRAWING

FIG. 1 is a schematic representation of vector pCytTs-EGFP-OPE described in Example 2. The EGFP expression cassette can be replaced by any gene of interest (e.g. SEAP, EPO, IFN). CMV: Cytomegalovirus promoter, puro: puromycin resistance marker; amp: ampicillin resistance marker; ColE1: bacterial origin of replication; SG prom: subgenomic promoter; ORIP, EBV origin of replication; EBNA1, EBV nuclear antigen 1; GFP: green fluorescence protein; nsp1-4: genes coding for the viral non-structural proteins containing the nsp 4 (Pro728Ser) mutation and the nsp2 (Gly153Glu) mutation that renders the replicase temperature sensitive and non-cytopathic.

### DETAILED DESCRIPTION OF THE INVENTION

The present invention is directed to compositions and methods that are useful, e.g., in the production of polypeptides and/or untranslated RNA molecules in host cells. The compositions and methods of the invention are also useful, e.g., for the isolation and/or purification of polypeptides, polypeptides and untranslated RNA molecules that are produced in host cells.

According to one aspect of the invention, nucleic acid molecules are provided. The nucleic acid molecules of the invention comprise: (A) a first polynucleotide element which encodes an RNA molecule, said RNA molecule comprising: (a) at least one cis-acting sequence element; (b) a first nucleotide sequence comprising a first open reading frame, said first open reading frame having a nucleotide sequence encoding an RNA-dependent RNA polymerase; and (c) at least one second nucleotide sequence selected from the group consisting of: (i) a second open reading frame encoding a polypeptide; (ii) a nucleotide sequence complementary to all or a part of the second open reading frame of (i); and (iii) a nucleotide sequence encoding an untranslated RNA molecule or complement thereof; wherein said second nucleotide sequence is operably linked to a promoter which is recognized by said RNA-dependent RNA polymerase; (B) a second polynucleotide element comprising an origin of replication; and (C) a third polynucleotide element encoding a replication initiation factor capable of recognizing said origin of replication.

According to another aspect of the invention, expression systems are provided. The expression systems of the invention comprise one or more nucleic acid molecules, wherein said one or more nucleic acid molecules comprise: (A) a first polynucleotide element which encodes an RNA molecule, said RNA molecule comprising: (a) at least one cis-acting sequence element; (b) a first nucleotide sequence comprising a first open reading frame, said first open reading frame having a nucleotide sequence encoding an RNA-dependent RNA polymerase; and (c) at least one second nucleotide sequence selected from the group consisting of: (i) a second open reading frame encoding a polypeptide; (ii) a nucleotide sequence complementary to all or a part of the open reading frame of (i); and (iii) a nucleotide sequence encoding an untranslated RNA molecule or complement thereof; wherein said second nucleotide sequence is operably linked to a promoter which is recognized by said RNA-dependent RNA polymerase; (B) a second polynucleotide element comprising an origin of replication; and (C) a third polynucleotide element encoding a replication initiation factor capable of recognizing said origin of replication.

In the expression systems of the invention, said first, second and third polynucleotide elements may each be on a separate nucleic acid molecule. Alternatively, said first, second and third polynucleotide elements may each be on a single nucleic acid molecule. In certain embodiments, said first and second polynucleotide elements will be on a single nucleic acid molecule while said third polynucleotide element is on a different nucleic acid molecule. In certain other embodiments, said first and third polynucleotide elements will be on a single nucleic acid molecule, while said second polynucleotide element is on a different nucleic acid molecule. In certain other embodiments, said second and third polynucleotide elements will be on a single nucleic acid molecule, while said first polynucleotide element is on a different nucleic acid molecule.

According to another aspect of the invention, recombinant host cells and in vitro cell cultures comprising recombinant host cells are provided. The recombinant host cells of the invention comprise: (A) a first polynucleotide element which encodes an RNA molecule, said RNA molecule comprising: (a) at least one cis-acting sequence element; (b) a first nucleotide sequence comprising a first open reading frame, said first open reading frame having a nucleotide sequence encoding an RNA-dependent RNA polymerase; and (c) at least one second nucleotide sequence selected from the group consisting of: (i) a second open reading frame encoding a polypeptide; (ii) a nucleotide sequence complementary to all or a part of the second open reading frame of (i); and (iii) a nucleotide sequence encoding an untranslated RNA molecule or complement thereof; wherein said second nucleotide sequence is operably linked to a promoter which is recognized by said RNA-dependent RNA polymerase; (B) a second polynucleotide element comprising an origin of replication; and (C) a third polynucleotide element encoding a replication initiation factor capable of recognizing said origin of replication.

According to another aspect of the invention, methods are provided for producing, or regulating the expression of, a polypeptide or untranslated RNA molecule. The methods according to this aspect of the invention comprise: (a) introducing a nucleic acid molecule or an expression system of the invention into a host cell to produce a recombinant host cell; and (b) culturing said recombinant host cell. The methods of the invention may further comprise recovering said polypeptide or untranslated RNA molecule.

According to another aspect of the invention, kits are provided comprising the nucleic acid molecules, expression systems and/or host cells of the invention.

In certain embodiments, said second open reading frame that is included within the compositions and methods of the invention is in a translatable format after one or more RNA-dependent RNA replication events. The expression “a translatable format” is intended to mean an RNA from which a protein can be made.

The RNA-dependent RNA polymerase that is included in, or used with, the compositions and methods of the invention may, in certain embodiments, be selected from the group consisting of: (a) a temperature-sensitive RNA-dependent RNA polymerase, (b) a non-cytopathic RNA-dependent RNA polymerase, and (c) a temperature-sensitive, non-cytopathic RNA-dependent RNA polymerase.

When the RNA-dependent RNA polymerase that is included in, or used with, the compositions and methods of the invention is temperature sensitive, the temperature-sensitive RNA-dependent RNA polymerase may have replicase activity at temperatures below 34° C. which is at least five fold, at least ten fold, at least twenty fold, at least thirty fold, at least forty fold, at least fifty fold, at least one hundred fold, or at least one thousand fold greater than the replicase activity exhibited at 34° C. or above. For example, the temperature-sensitive RNA-dependent RNA polymerase may have replicase activity at 34° C. that is at least five fold greater than the replicase activity exhibited at 29° C. The temperature-sensitive RNA-dependent RNA polymerase may, in certain embodiments, exhibit no or undetectable replicase activity (using methods of detection known in the art) at 34° C. or above.

The RNA-dependent RNA polymerase that is included in, or used with, the compositions and methods of the invention may, in certain embodiments, be of viral origin. For example, the RNA-dependent RNA polymerase may be of alphaviral origin. In specific embodiments of the invention, the RNA-dependent RNA polymerase is derived from a Sindbis virus, a Semliki Forest virus or an Aura virus. The RNA-dependent RNA polymerase may be derived from a virus selected from the group consisting of Bebaru virus, Cabassou virus, Chikungunya virus, Easter equine encephalomyelitis virus, Fort Morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Venezuelan equine encephalomyelitis virus (VEE), and Ross River virus.

The origin of replication that is included in, or used with, the nucleic acid molecules, the expressions systems, compositions and methods of the invention may be derived from a prokaryotic organism, a eukaryotic organism (e.g., a yeast, insect or mammal), or a virus. For example, the origin of replication may be derived from a DNA virus, e.g., a DNA virus that allows for episomal replication. The origin of replication may be derived from a DNA virus selected from the group consisting of Herpesvirus, Epstein-Barr virus (EBV), Papillomavirus, Polyomavirus, Adenovirus, and Hepadnavirus. In a specific embodiment, the origin of replication is oriP, derived from EBV.

The replication initiation factor that is included in, or used with, the compositions and methods of the invention is, in certain embodiments, capable of operating as a plasmid maintenance factor. As used herein, the term “plasmid maintenance factor” is intended to mean a factor, which supports the distribution of the episomes to the daughter cells upon cell division. In the case of EBNA-1, this is achieved by the binding of the protein to the episome and, at the same time, to the chromosome, thereby leading to proper distribution of the episomes to the daughter cells. The replication initiation factor may be derived from a prokaryotic organism, a eukaryotic organism (e.g., a yeast, insect or mammal), or a virus. For example, the replication initiation factor may be derived from a DNA virus, e.g., a DNA virus that allows for episomal replication. The replication initiation factor may be derived from a DNA virus selected from the group consisting of Herpesvirus, Epstein-Barr virus (EBV), Papillomavirus, Polyomavirus, Adenovirus, and Hepadnavirus. In a specific embodiment, the replication initiation factor is the EBNA-1 protein, derived from EBV.

The origin of replication and the replication initiation factor that are included in, or used with, the compositions and methods of the invention may, in certain embodiments, be derived from the same organism or the same virus. Alternatively, the origin of replication and the replication initiation factor may be derived from different organisms or viruses.

The nucleic acid molecules and expression systems of the invention may further comprise a fourth polynucleotide element, wherein said fourth polynucleotide element comprises a selection marker. Any selection marker known in the art may be used, including, eg., markers that confer resistance to antibiotics. An exemplary selection marker is one that confers resistance to puromycin. Alternatively, selection markers may be used that confer resistance to hygromycin, gpt, neomycin, zeocin, ouabain, blasticidin, or bleomycin, or markers such as DHRF, hisD, trpB, or glutamine synthetase.

The second open reading frame that is included in, or used with, the compositions and methods of the invention may encode, in certain embodiments, any polypeptide of interest. For example, the second open reading frame may encode, e.g., a cytokine, a lymphokine, a tumor necrosis factor, an interferon (e.g., beta-interferon, including human beta-interferon), a toxic polypeptide, or a prodrug.

The second open reading frame that is included in, or used with, the compositions and methods of the invention may encode, in certain embodiments, an untranslated RNA molecule. Exemplary untranslated RNA molecules include, e.g., an antisense RNA molecule, a tRNA molecule, an rRNA molecule and a ribozyme. Other useful untranslated RNA molecules are known in the art.

Thus, in certain exemplary embodiments, the invention provides compositions and methods which allow for the production of high amounts of specific RNA molecules produced in host cells upon their transient transfection with said polynucleotides. This high production results, e.g., from the use of a self-replicating episomal DNA plasmid combined with an RNA-dependent RNA polymerase, which amplifies the mRNA of interest.

Expression, using the compositions and methods of the invention, may be regulated precisely, if necessary. Such precise regulation can be achieved, e.g., from the use of a temperature-sensitive RNA-dependent RNA polymerase which only replicates RNA molecules to form additional RNA molecules at permissive temperatures.

According to the present invention, polypeptides and/or untranslated RNA molecules may be produced in transient transfection experiments due to the use of nucleic acid molecules comprising an origin of replication and DNA encoding a protein (a factor or a DNA-dependent replicase) that can recognize the origin of replication, thereby resulting in replication of the plasmid.

The expression systems of the invention, in certain embodiments, comprise alphavirus DNA vectors that can be used to create transiently transfected cell lines. Said alphavirus DNA vectors may carry genes encoding a non-cytopathic replicase and viral non-structural proteins, thereby being able to produce high amounts of polypeptide. An induction of expression is achieved when the activity of the temperature-sensitive replicase is switched on by reducing the incubation temperature of the transfected cells from 37° C. to a temperature lower than 34° C. Protein expression in the host at 37° C. is, preferably, below the level of detection.

### DEFINITIONS

The following definitions are provided to clarify the subject matter, which the inventors consider to be the present invention.

As used herein, a nucleic acid molecule is a sequence of contiguous nucleotides (riboNTPs, dNTPs or ddNTPs, or combinations thereof) of any length which may encode a full-length polypeptide or a fragment of any length thereof, or which may be non-coding. As used herein, the terms “nucleic acid molecule,” and “polynucleotide,” “polynucleotide construct,” and “polynucleotide element” may be used interchangeably.

As used herein, the term “alphavirus” refers to any of the RNA viruses included within the genus Alphavirus. Descriptions of the members of this genus are contained in Strauss and Strauss, Microbiol. Rev., 58:491-562 (1994). Examples of alphaviruses are selected from the group comprising Aura virus, Bebaru virus, Cabassou virus, Chikungunya virus, Easter equine encephalomyelitis virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Sindbis virus (SIN), Semliki forest virus (SFV), Venezuelan equine encephalomyelitis virus (VEE), and Ross River virus.

As used herein, the term “purified” used in reference to a molecule means that the concentration of the molecule being purified has been increased relative to molecules associated with it in its natural environment. Naturally associated molecules include polypeptides, nucleic acids, lipids and sugars but generally do not include water, buffers, and reagents added to maintain the integrity or to facilitate the purification of the molecule. For example, even if mRNA is diluted with an aqueous solvent during oligo dT column chromatography, mRNA molecules are purified by this chromatography if naturally associated nucleic acids and other biological molecules do not bind to the column and are separated from the subject mRNA molecules.

As used herein, the term “isolated” used in reference to a molecule means that the molecule has been removed from its native environment. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need to be “purified.”

As used herein, the phrase “low or undetectable,” when used in reference to gene expression level, refers to a level of expression which is either significantly lower than that seen when the gene is maximally induced (e.g., at least five fold lower) or is not readily detectable by the methods used in the following examples section.

As used herein, the phrase “individual” refers to multicellular organisms and includes both plants and animals. Preferred multicellular organisms are animals, more preferred are vertebrates, even more preferred are mammals, and most preferred are humans.

As used herein, the term “pCytTs” means any temperature sensitive, non-cytopathic inducible alphaviral expression system containing a functional promoter to drive the transcription of mRNA from the nucleic acid molecule of the invention. Examples for such expression systems or vectors may be, e.g., the vector pCytTs (SEQ ID NO: 1) containing a RSV promoter or the vector pCytTs2.1 (SEQ ID NO: 2) containing a CMV promoter. When combinatorial constructs with pCytTs are mentioned in the Examples, they generally contain a CMV promoter, even if the abbreviation of the combinatorial constructs does—for the sake of simplicity—only refer to pCytTs and not to pCytTs2.1.

As used herein, the phrase “cis-acting” sequence refers to nucleic acid sequences to which a replicase binds to catalyze the RNA-dependent replication of RNA molecules. These replication events result in the replication of the full-length and partial RNA molecules and, thus, the alphavirus subgenomic promoter is also a “cis-acting” sequence. Cis-acting sequences may be located at or near the 5′ end, 3′ end, or both ends of a nucleic acid molecule, as well as internally.

As used herein, the phrase “RNA-dependent RNA polymerase” refers to a polymerase which catalyzes the production of an RNA molecule from another RNA molecule. This term is used herein synonymously with the term “replicase.”

As used herein, the phrase “non-infective packaged RNA molecules” refers to packaged RNA molecules which will essentially undergo only one round of host cell infection and are not pathogenic. These molecules are thus “infective” but only for a single infectious entry into a host cell and are not capable of reproducing to form additional infectious particles.

As used herein, the term “transcription” refers to the production of RNA molecules from DNA templates catalyzed by RNA polymerases.

As used herein, the phrase “RNA-dependent RNA replication event” refers to processes which result in the formation of an RNA molecule using an RNA molecule as a template.

As used herein, the term “vector” refers to an a polynucleotide construct, typically a plasmid or a virus, used to transmit genetic material to a host cell. Preferably, the term “vector” as used herein refers to an agent such as a plasmid, and even more preferably to a circular plasmid, A vector as used herein may be composed of either DNA or RNA. Preferably, a vector as used herein is composed of DNA.

As used herein, the term “heterologous sequence” refers to a second nucleotide sequence present in a vector of the invention. The term “heterologous sequence” also refers to any amino acid or RNA sequence encoded by a heterologous DNA sequence contained in a vector of the invention. Heterologous nucleotide sequences can encode polypeptides or RNA molecules normally expressed in the cell type in which they are present or molecules not normally expressed therein (e.g., Sindbis structural proteins).

As used herein, the phrase “untranslated RNA” refers to an RNA sequence or molecule which does not contain an open reading frame or encodes an open reading frame, or portion thereof, but in a format in which an amino acid sequence will not be produced (e.g., no initiation codon is present). Examples of such molecules are tRNA molecules, rRNA molecules, and ribozymes. Antisense RNA may be untranslated but, in some instances antisense sequences can be converted to a translatable sense strand from which a polypeptide is produced.

As used herein, the phrase “temperature-sensitive” refers to an enzyme which readily catalyzes a reaction at one temperature but catalyzes the same reaction slowly or not at all at another temperature. An example of a temperature-sensitive enzyme is the replicase protein encoded by the pCytTs vector, which has readily detectable replicase activity at temperatures below 34° C. and has low or undetectable activity at 37° C.

As used herein, the phrase “permissive temperature” refers to temperatures at which an enzyme has relatively high levels of catalytic activity.

As used herein, the phrase “restrictive temperature” refers to temperatures at which an enzyme has low or undetectable levels of catalytic activity. Both “hot” and “cold” sensitive mutants are known and, thus, a restrictive temperature may be higher or lower than a permissive temperature.

As used herein, the term “recombinant host cell” refers to a host cell into which one ore more nucleic acid molecules of the invention have been introduced.

As used herein, the term “replication initiation factor” refers to a protein or a DNA sequence encoding such a protein that is able to recognize an origin of replication leading to replication of plasmids containing this origin of replication.

As used herein, the term “origin of replication” refers to a DNA sequence that is recognized by a replication initiation factor or a DNA replicase leading to replication of a plasmid containing the origin of replication. The expression “recognized by a replication initiation factor” is intended to mean that a replication initiation factor can physically interact with all or a portion of an origin of replication sequence, thereby causing or stimulating molecular mechanisms that ultimately cause all or a portion of the DNA molecule comprising the origin of replication to be replicated.

As used herein the term “inducible” means that in an inactive state, the basal activity of a system should be minimal, while in the active state high levels of gene expression should be rapidly inducible.

When the terms “one,” “a,” or “an” are used in this disclosure, they mean “at least one” or “one or more,” unless otherwise indicated.

Nucliec Acid Molecules and Expression Systems

Certain preferred embodiments of the present invention are based on the vectors used in the previously described pCytTs expression system (WO 99/50432, the disclosure of which is hereby incorporated by reference in its entirety). These vectors are constitutively transcribed in host cells to produce mRNA molecules having two open reading frames. These open reading frames, which may or may not be produced from the same nucleic acid molecule, encode a temperature-sensitive replicase and a heterologous gene of interest. The first open reading frame is translated to produce an RNA-dependent RNA polymerase. The second open reading frame, encoding all or part of one or more polypeptides of interest, is not translated until after at least one RNA-dependent RNA replication event.

The nucleic acid molecule of the present invention comprise, inter alia, at least one second nucleotide sequence. The second nucleotide sequence is preferably operably linked a promoter which is recognized by an RNA-dependent RNA polymerase. The second nucleotide sequence, in certain embodiments, may comprise a second open reading frame, or other nucleotide sequence of interest, as well as other elements. For instance, the second nucleotide sequence may comprise a 5′ promoter which is capable of initiating synthesis of RNA in vivo, 5′ and/or 3′ sequences enabling replication of the RNA molecule (5′ and 3′ cis acting sequence elements), as well as a sequence of interest which is translatable only after at least one replication event. Replication is catalyzed by an RNA-dependent RNA polymerase which is encoded alternatively on the same or on a different mRNA molecule. The sequence of interest may be encoded in sense, plus (+), orientation downstream of a viral RNA promoter. Translation of the coding sequence of the gene of interest is inhibited by a 5′ sequence which, in the case of the single-vector system, will generally be the replicase sequence. In the multiple-vector system, a 5′ sequence can inhibit translation by having one or more short open reading frames with associated stop codons which lead to the detachment of ribosomes. Similarly, any sequence which inhibits the traveling or binding of ribosomes to the sequence of interest can be used as a 5′ sequence which inhibits translation (Voet and Voet, BIOCHEMISTRY, John Wiley & Sons, Inc. (1990)).

Another method for preventing translation of nucleotide sequences in most biological systems involves the insertion of the sequence in an antisense direction. This method of inhibiting translation is based on the principle that translation will generally only occur after the replication of this minus (−) strand RNA into a plus strand having an open reading frame in a sense orientation. The translated sense strand is formed by RNA replication and serves as a template for ribosomes and protein synthesis. As shown previously (WO 99/50432) production of amino acid sequences can occur even when the gene of interest is inserted into the DNA molecule in an orientation which will result in the formation of antisense RNA sequence 3′ to the subgenomic promoter. Thus, the second open reading frame may also comprise a sequence complementary to all or part of the second open reading frame described above and expression of the encoded amino acid sequence will still occur. When the production of an untranslated antisense RNA sequence is desired, the RNA molecule can be designed so that it will not serve as a template for protein synthesis. For example, the RNA can be designed so that an initiation codon is not present.

The second nucleotide sequence may alternatively or additionally comprise a nucleotide sequence encoding an untranslated RNA molecule or complement thereof. For example, RNA molecules directly produced by transcription of a DNA sequence of the invention may encode RNA sequences which are neither translated nor present in an antisense orientation. Examples of such untranslated RNA molecules include tRNA molecules, rRNA molecules, and ribozymes. A considerable number of ribozyme sequences with defined catalytic activities are known in the art (see, e.g., Brown, J., Nucleic Acids Res. 26:351-352 (1998); Xie, Y. et al., Proc. Natl. Acad. Sci. USA 94:13777-13781 (1997); Lavrovsky. Y et al., Biochem. Mol. Med. 62:11-22 (1997); Chapman, K. and Szostak, J., Chem. Biol. 2:325-333 (1995)). Further, ribozymes have been used to “knockout” the expression of a specific gene in eukaryotic cells as part of a ribozyme-mediated, message deletion strategy (Xie, Y. et al., Proc. Natl. Acad. Sci. USA 94:13777-13781 (1997)). Additionally, alphaviral replicons have been used to express a functional ribozyme in mammalian cells (Smith S. et al., J. Virol. 71:9713-9721 (1997)). The regulated expression of such ribozymes, and other untranslated RNA molecules, is thus within the scope of the present invention.

The nucleic acid molecules and expression systems of the invention also comprise a second polynucleotide element comprising an origin of replication. Origins of replication that can be used with the present invention include, e.g., those that are derived from prokaryotic organisms, eukaryotic organisms (e.g., yeasts, insects, or mammals) and/or viruses. For example, an origin of replication that may be used with the present invention is one that is derived from a DNA virus such as, e.g., a Herpesvirus, a Papillomavirus, an Adenovirus, or a Hepadnavirus. In certain embodiments, the origin of replication that is used with the invention is a DNA virus that allows for episomal replication. In a preferred embodiment, the origin of replication is derived from Epstein-Barr virus (EBV), such as, e.g., oriP.

The nucleic acid molecules and expression systems of the invention also comprise a third polynucleotide element encoding a replication initiation factor. The replication initiation factor will be capable of recognizing the origin of replication that is included within the nucleic acid molecule or expression system. The third polynucleotide element of the invention may encode, e.g., a replication initiation factor derived from prokaryotic organism, eukaryotic organism (e.g., yeasts, insects, and/or mammals) and/or viruses. For example, a replication initiation factor that may be used with the present invention is one that is derived from a DNA virus such as, e.g., a Herpesvirus, a Papillomavirus, an Adenovirus, or a Hepadnavirus. In certain embodiments, the replication initiation factor that is used with the invention is capable of operating as a plasmid maintenance factor. In a preferred embodiment, the replication initiation factor is derived from Epstein-Barr virus (EBV), such as, e.g., the EBNA-1 protein, or a portion thereof.

The replication initiation factor (e.g., a nucleotide sequence that encodes a replication initiation factor) and the origin of replication that are included within the nucleic acid molecules and expression systems of the present invention may be derived from the same organism or from the same virus. Alternatively, the replication initiation factor and the origin of replication may be derived from different organisms or from different viruses.

In an exemplary embodiment of the invention, nucleic acid molecules are provided, which are constructed by introducing defined Epstein-Barr virus (EBV) sequences into vectors such as, e.g., those included within the pCytTs system. The resulting exemplary nucleic acid molecules of the invention are capable of replicating as non-integrated autonomous episomal molecules in the transformed host cells.

The defined sequences, which may be introduced into the vectors of the pCytTs system, comprise, e.g., either one or two elements of Epstein-Barr virus, (OriP alone or together with EBNA-1 gene) which permit plasmid maintenance. The origin of replication OriP is a cis-acting sequence and needs to be inserted into the plasmid vector sequence. Plasmids containing this origin of replication are able to be maintained in cells expressing the replication initiation factor which recognizes the origin of replication; one of these factors is EBNA-1. EBNA-1 gene function can be provided in cis by introducing the sequence into the same plasmid or can be provided in trans by co-transfection with a second replicating or non-replicating plasmid as well as by providing it from stably transduced cell lines expressing EBNA-1 gene from an integrated copy. Furthermore, in certain embodiments, more than one copy of the sequence expressing the replication initiation factor, e.g. EBNA-1, is provided. For example, EBNA-1 gene function may be provided on one or more plasmids, wherein none, some or all of those plasmids may additionally comprise the origin of replication, and which plasmids are used for transfecting cell lines, preferably cell lines that have the sequence of EBNA-1 stably integrated within its genome. The presence of both OriP and EBNA1 sequences in the same plasmid renders replication less dependent on the host cell type. (Reisman, D. et al., Mol. Cell Biol. 5: 1822-1832 (1985); Yates, J. L. et al., Nature 313:812-815 (1985); U.S. Pat. No. 4,686,186). U.S. Pat. No. 4,686,186 describes the transfection of cells with a single plasmid containing the EBV OriP, the EBNA-1 gene and a gene encoding a protein of interest.

Although such cell lines have the advantage of stable long-term expression of the replication initiation factor and durable support of replication and maintenance of OriP containing plasmids, there are not many EBNA-1 expressing cell lines commercially available (ATCC: 293HEK-EBNA1 and CV1-EBNA1). Alternatively, plasmids that already carry the EBNA-1 gene and the gene of interest in cis on the same episome are used to transfect cells and commercial vectors such as pCEP4 (Invitrogen) are available. However, current vectors designed for constitutive expression of polypeptides carrying OriP and EBNA-1 on the episomal construct in cis and wherein the expression is not regulatable or inducible, are not applicable in cases where cell-toxic polypeptides need to be expressed.

To our knowledge, an inducible expression system having the features of a self-replicating DNA episome and that allows the expression of a polypeptide or untranslated RNA sequence of interest from a self-replicating RNA molecule, triggered by a simple temperature shift, has not been described.

Certain embodiments of the invention are directed to nucleic acid molecules which are transcribed to produce a mRNA molecule having two open reading frames, e.g., one open reading frame which encodes a replicase, and another open reading frame which encodes a nucleotide sequence of interest. The nucleic acid molecules of the invention may contain a promoter sequence which drives transcription to produce mRNA molecules having coding sequences of both open reading frames. The mRNA sequences of the first open reading frame are preferably translated to produce a replicase required for the expression of the RNA sequences of the second open reading frame. The second open reading frame preferably encodes one or more polypeptides of interest. In addition, the vectors contain an origin of replication and a DNA polymerase recognizing the origin of replication.

Further, once the first mRNA molecule has been transcribed from the DNA vector, additional RNA-dependent RNA replication events may occur to amplify the first mRNA sequence and to produce RNA molecules with strand polarity which is the opposite of the first mRNA sequence.

The second open reading frame of the nucleic acid molecules of the invention will preferably only be expressed after partial replication of a full-length RNA molecule. This partial replication of the full-length RNA molecules is driven by a promoter sequence composed of RNA (e.g., an alphaviral subgenomic promoter sequence). In addition, the plasmid encoding the replicase and the gene of interest will preferably replicate during division of the host cell due to the presence of origin of replication and a protein factor recognizing the origin of replication.

While the gene of interest may be encoded by the same RNA molecule as the RNA-dependent RNA replicase protein, this gene may also be encoded by a separate RNA molecule. Thus, the invention further provides both single- and multiple-vectors systems for expressing a gene of interest. Moreover, while all plasmids used preferably contain an origin of replication, the gene encoding the replication initiation factor may be on a separate plasmid or stably integrated into the genome of the host cell.

In a single-vector system of the invention, sequences encoding the first open reading frame, the second nucleotide sequence are components of the same nucleic acid molecule, the origin of replication and the initiation factor. Thus, all of the components required for regulated expression of the gene of interest and plasmid amplification are contained in a single nucleic acid molecule (i.e., DNA or RNA).

In a multiple-vector system of the invention, sequences encoding the first open reading frame, or sub-portions thereof, and the second nucleotide sequence are components of different nucleic acid molecules. These multiple-vector systems thus may comprise two or more nucleic acid molecules. For example, the replicase and the gene of interest can each be encoded by different nucleic acid molecules. However, all vectors may contain an origin of replication.

While any functional promoter can be used to drive the transcription of mRNA from the nucleic acid molecule of the invention, the promoter is preferably a constitutive RNA polymerase II promoter (e.g., Rous Sarcoma Virus (RSV), cytomegalovirus (CMV), simian virus 40 (SV40), myeloproliferative sarcoma virus (MPSV), glucocorticoid, metallothionein, Herpes simplex virus thymidine kinase (HSVTK), human immune deficiency (HIV), mouse mammary tumor virus (MMTV), human polyomavirus BK (BKV), or Moloney murine leukemia virus (MuLV) promoter). Additional promoters suitable for use in the practice of the present invention are known in the art (see, e.g., Lee, A. et al., Mol. Cells. 7:495-501 (1997)).

The nucleic acid molecules of the invention may further comprise a fourth polynucleotide element, wherein said fourth polynucleotide element comprises a selection marker. The selection marker may facilitate the cloning and amplification of the vector sequences in prokaryotic and eukaryotic organisms. In certain embodiments, the selection marker will confer resistance to a compound or class of compounds, such as an antibiotic. An exemplary selection marker that can be used with the nucleic acid molecules and expression systems of the present invention is one that confers resistance to puromycin. Alternatively, selection markers may be used that confer resistance to hygromycin, gpt, neomycin, zeocin, ouabain, blasticidin, or bleomycin, or markers such as DHRF, hisD, trpB, or glutamine synthetase.

The pCytTs vector contains an ampicillin resistance marker for positive selection in bacterial host cells and an E. coli origin of replication (i.e., ColE1). A considerable number of sequences encoding additional selection markers and origins of replication are known in the art (see, e.g., Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS IN MOLECULAR BIOLOGY, John H. Wiley & Sons, Inc. (1997)).

The nucleic acid molecules and expression systems of the invention comprise a first nucleotide sequence comprising a first open reading frame, said first open reading frame having a nucleotide sequence encoding a replicase, e.g., an RNA-dependent RNA polymerase. The first nucleotide sequence may, in certain embodiments, also comprise 5′ and 3′ cis-acting sequences, and junction sequences containing a subgenomic promoter.

The RNA-dependent RNA polymerases that can be used with the present invention may be of viral origin. The RNA-dependent RNA polymerase may be derived, e.g., from virus selected from the group consisting of: Bebaru virus, Cabassou virus, Chikungunya virus, Easter equine encephalomyelitis virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Venezuelan equine encephalomyelitis virus (VEE), and Ross River virus. In a preferred embodiment, the RNA-dependent RNA polymerase of the invention is derived from an alphavirus such as, e.g., a Sindbis virus, a Semliki Forest virus, and/or an Aura virus.

In certain embodiments of the invention, the RNA-dependent RNA polymerase that is encoded by the first open reading frame of the nucleic acid molecules of the invention will be a non-cytopathic RNA-dependent RNA polymerase. The RNA-dependent RNA polymerase may, alternatively, be a temperature sensitive RNA-dependent RNA polymerase. In other embodiments, the RNA-dependent RNA polymerase may be a non-cytopathic, temperature-sensitive RNA-dependent RNA polymerase.

When the RNA-dependent RNA polymerase that is encoded by the first open reading frame of the nucleic acid molecules of the invention is a temperature-sensitive RNA-dependent RNA polymerase, the RNA-dependent RNA polymerase may, e.g., have replicase activity at temperatures below 34° C. and low or undetectable replicase activity at 34° C or above. In certain embodiments, the temperature sensitive RNA-dependent RNA polymerase will be one that has replicase activity at 34° C. which is at least five fold lower than the replicase activity exhibited at 29° C.

Preferred mutations which confer a non-cytopathic phenotype are those that are in the nsp2 gene (e.g., the proline residue at position 726 is replaced with a serine or a leucine residue). Mutations are known in the art which render the replicase protein non-cytopathic (Weiss et al., J. Virol. 33:463-474 (1980); Dryga et al., Virology 228:74-83 (1997); Agapov et al., PNAS 95:12989-12994 (1998)). In addition, mutations may be introduced into the nuclear localization signal of an alphaviral replicase which diminish toxicity of the replicon by, e.g., modifying the distribution of the replicase-encoding sequence between the cytoplasm and the nucleus. (Rikkonen et al., Virology 218:352-361 (1996)).

Mutations which render a replicase non-cytopathic may be introduced by a number of means, including site directed mutagenesis. Additional mutations that render an RNA-dependent RNA polymerase non-cytopathic can be identified by, e.g., random mutagenesis and screening using methods and techniques that are well-known in the art. As will be understood by persons having ordinary skill in the art, the identification of additional mutations that render an RNA-dependent RNA polymerase non-cytopathic, temperature sensitive, or both, does not require an ability to predict the structural or functional consequences of any particular mutation.

About 20 years ago Weiss et al. (Weiss, B. et al., J. Virol. 33:463-474 (1980)) established a persistently infected culture of BHK cells. The mutation responsible for this phenotype has been recently identified (Dryga, S. A. et al., Virology 228:74-83 (1997)). Another mutation allowing the regulation of the mRNA transcription via temperature shifts was identified by Burge and Pfefferkom (Burge, B. W. & Pfefferkom, E. R., Virology 30:203-214 (1966)) and described in more detail by Xiong et al. (Xiong, C. et al., Science 243:1188-1191 (1989)).

Temperature sensitivity (ts) may be conferred, for example, by the introduction of a mutation in the nsp4 gene of the replicase. Preferably, mutations which confer a temperature-sensitive phenotype upon replicase activities are in a protein in complementation group F (Lemm et al., J. Virol. 64:3001-3011 (1990)). For example, a temperature-sensitive phenotype may be conferred by changing Gly 153 of nsp4 to Glu. Additionally, any other mutation which renders replicase activity temperature-sensitive can be used in the practice of the invention. Methods for creating and identifying new temperature-sensitive mutants are described by Pfefferkorn (Burge and Pfefferkorn, Virol. 30:204-213(1966); Burge and Pfefferkorn, Virol. 30:214-223 (1966)). Other methods will be appreciated by those of ordinary skill in the art.

While most temperature-sensitive mutants are “hot” sensitive, “cold” sensitive mutations are also known (see, e.g., Schwer, B. et al., Nucleic Acids Res. 26:803-809 (1998), Mathe, E. et al., J. Cell Sci. 111:887-896 (1998), Doedens, J. et al., J. Virol. 71:9054-9064 (1997), Patterson, B. et al., J. Biol. Chem. 272:27612-27617 (1997)). The temperature-sensitive replicase may be “cold” or “hot” sensitive and thus will catalyze RNA replication only at temperatures either above or below restrictive temperatures. In one embodiment, RNA replication occurs at detectable levels only at temperatures lower than 34° C. In contrast to all previously known regulatable DNA expression systems, the basal level of expression in recombinant host cells containing the pCytTs vector in the inactive state at 37° C. is below the level of detection using standard methods (e.g., those used in the following examples) even under condition of transient transfection. This low level of expression is apparent from the data presented in Table 1.

The production of additional temperature sensitive and/or non-cytopathic replicases has been described in the art. (Lundstrom et al., Gene Ther. Mol. Biol. 4:23-31 (1999), Lundstrom et al., Histochem. Cell. Biol. 115:83-91 (2001)). Moreover, with respect to alphaviruses, the results obtained in one type of alphavirus can often be used to predict the results obtained in other alphaviruses. That is, the identification of mutations that render a replicase temperature-sensitive and/or non-cytopathic in one alphavirus type may be used to identify or create a similar temperature-sensitive and/or non-cytopathic replicase in a different alphavirus. This ability is related, in part, to the high level of sequence identity observed among alphavirus genomes, especially in their replicase-encoding sequences. (Weaver et al., J. Virol 71:613-623 (1997), Smyth et al., J. Virol. 71:818-823 (1997), Kuhn et al., J. Virol. 70:7900-7909 (1996)).

In certain embodiments, the first nucleotide sequence (comprising a first open reading frame encoding an RNA-dependent RNA polymerase) and the second nucleotide sequence (comprising, e.g., a second open reading frame encoding a polypeptide) are contained on two separate nucleic acid molecules. In such an instance, the second nucleotide sequence may carry both cis-acting sequences and a 5′ region which inhibits translation of the sequence of interest. The first nucleotide sequence can also be encoded by a nucleic acid molecule which is different than the second nucleotide sequence. Replication and translation of the second nucleotide sequence in this multi-vectors system can be regulatable by temperature if a temperature sensitive RNA-dependent RNA polymerase is used.

Additional mutations that render an RNA-dependent RNA polymerase non-cytopathic, temperature-sensitive, or both, can be identified by, e.g., random mutagenesis and screening using methods and techniques that are well-known in the art. As will be understood by persons having ordinary skill in the art, the identification of additional mutations that render an RNA-dependent RNA polymerase non-cytopathic, temperature sensitive, or both, does not require an ability to predict the structural or functional consequences of any particular mutation. The identification of an RNA-dependent RNA polymerase that is non-cytopathic, temperature-sensitive, or both, only involves, e.g., the generation of random mutations and the functional testing of the replicases that are encoded from the mutated nucleic acid molecules. Such screening methods are routine in the art.

The nucleic acid molecules and expression systems of the invention can be also used to express of more than one gene of interest. For example, recombinant host cells can be transfected with more than one nucleic acid molecule of the invention wherein one nucleic acid molecule encodes both the replicase and a polypeptide of interest and additional nucleic acid molecules may encode additional polypeptides of interest. Similarly, when mutations conferring non-cytopathicity and temperature sensitivity are both used, genes encoding polypeptides having suitable mutations (e.g., Pro 726 Ser in nsp2 and Gly 153 Glu in nsp4) may be on separate nucleic acid molecules. Additional variations would be apparent to those skilled in the art.

The nucleic acid molecules of the invention may also contain packaging signals which direct the packaging of RNA molecules into viral particles. These RNA molecules can be packaged in the presence of wild-type virus or defective helper virus RNA. A significant improvement was made with the development of defective helper RNA molecules (Bredenbeek, P. et al., J. Virol. 67:6439-6446 (1993)). These RNA molecules contain cis-acting sequences, required for replication of the full-length transcription product, and subgenomic RNA promoter sequences which drive the expression of the structural protein genes. For example, in cells containing both RNA molecules with packaging signals and the defective helper virus RNA, alphaviral non-structural proteins allow for replication and amplification of the defective helper virus RNA sequences which are translated to produce virion structural proteins. Since the helper virus RNA lacks packaging signals, these molecules are not packaged into assembled virions. Thus, virion particles produced in this way contain essentially only RNA sequences encoding the gene of interest and, generally, other sequences required for temperature-sensitive regulation of gene expression. These non-infective packaged RNA molecules do not contain sequences encoding virion structural proteins and, thus, undergo only one round of host cell infection and are not pathogenic.

Non-infective packaged RNA molecules can be used to infect a culture of suitable host cells simply by adding the particles to culture medium containing these cells. The preparation of non-infective alpahviral particles is described in a number of sources, including “Sindbis Expression System”, Version C, (Invitrogen Catalog No. K750-1).

One exemplary application of this system is directed to the temperature-dependent production of non-infective, packaged RNA molecules. These packaged RNA molecules may be produced by a number of means including using recombinant host cells containing two different nucleic acid molecules (e.g., a nucleic acid molecule of the invention and a nucleic acid molecule encoding a helper virus RNA sequence). For example, one of these nucleic acid molecules will encode, e.g., an RNA molecule which contains packaging signal sequences, sequences encoding a non-cytopathic, temperature-sensitive replicase, and the gene of interest. The other nucleic acid molecule will contain sequences encoding alphaviral structural proteins downstream from an alphavirus subgenomic promoter. Using such a system, viral particles containing only RNA molecules with packaging signals will be produced at permissive temperatures in recombinant host cells. This is so because alphaviral structural proteins will only be produced at a permissive temperature. Additional variations of the above would be apparent to one skilled in the art.

The second nucleotide sequence that is included within the nucleic acid molecules and expression systems of the invention may be, e.g., a second open reading frame encoding a polypeptide. Such a second open reading frame may, alternatively or interchangeably, be referred to as a nucleotide sequence of interest. A wide variety of nucleotide sequences of interest can be expressed by the nucleic acid molecules and expression systems of the invention. These sequences include, but are not limited to, sequences encoding lymphokinies, cytokines, toxins, enzymes, prodrug converting enzymes, antigens which stimulate immune responses, single chain antibodies, polypeptides which stimulate or inhibit immune responses, tumor necrosis factors, and various proteins with therapeutic applications (e.g., growth hormones and regulatory factors).

In certain embodiments, the second open reading frame (the second nucleotide sequence) of the invention may encode, e.g., a cytokine or lymphokine (e.g., β-interferon). Hematopoiesis is regulated by lymphokines and cytokines which stimulate the proliferation and/or differentiation of various hemopoietic cells. Representative examples of cytokines and lymphokines include interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin- 10 (IL-10), interleukin-11 (IL-11), interleukin- 12 (IL-12), interleukin-13 (IL-13), interleukin-14 (IL-14), interleukin-15 (IL-15), interleukin-16 (IL-16), interleukin-17 (IL-17), granulocyte colony stimulating factor (G-CSF), granulocyte-macrophage colony stimulating factor (GM-CSF), macrophage colony stimulating factor (M-CSF), and interferons.

In certain embodiments, the second open reading frame (the second nucleotide sequence) of the invention may encode secreted enzymes (e.g., secreted alkaline phosphatase), cytoplasmic enzymes (e.g., green fluorescent protein), or any number of other proteins with therapeutic applications (e.g., human insulin, human coagulation Factor VIII).

The nucleic acid molecules and expression systems of the invention can also be used to express heterologous sequence encoding cytotoxic polypeptides. Cytotoxic polypeptides act to directly or indirectly inhibit cellular growth or metabolism. Representative examples of toxins include Shigella toxin, ricin, Diphtheria toxin, Cholera toxin, Pseudomonas exotoxin A, and Herpes simplex virus thymidine kinase (HSVTK). Within other embodiments of this invention, the heterologous sequence encodes a prodrug converting enzyme. A prodrug converting enzyme activates a compound with little or no cytotoxicity into a toxic product. Representative examples are HSVTK, alkaline phosphatase, guanine phosphoribosyl transferase, and penicillin-V amidase. Examples of both cytotoxic polypeptides and prodrug converting enzymes are discussed in numerous sources including PCT/US97/06010, EP 0716148, and WO 96/17072. In addition, a vast array of signaling molecules and membrane proteins are toxic if expressed at high levels. All these molecules may be suitable for expression using the system of the present invention.

The second nucleotide sequence of the nucleic acid molecules and expression systems of the invention may also be a nucleotide sequence encoding an untranslated RNA molecule or complement thereof. Exemplary untranslated RNA molecules include, e.g., antisense sequences, RNase P targeted sequences which induce gene down-regulation, and ribozymes. Smith S. et al. (J. Virol. 71:9713-9721 (1997)) describes alphaviral vectors used to express ribozyme sequences.

The nucleic acid molecules and expression systems of the invention can also be used to express virtually any polypeptide, including ones which have not as yet been identified but are encoded by nucleotide sequences contained in, for example, cDNA libraries or host cell chromosomes. Example of such polypeptides include secreted proteins and proteins from various cellular compartments. Heterologous sequences expressed by the vectors of the invention can encode polypeptides and RNA molecules from non-human species (e.g., other mammals, plants, fungi, bacteria or viruses). These heterologous sequences may further encode viral membrane proteins (e.g., HIV gp160) or viral polyproteins (e.g., Sindbis structural proteins).

Nucleotide sequences may be added to the nucleic acid molecules and vectors of the invention which result in the production of a fusion protein. For example, such sequences can encode amino acids sequences which are fused to a protein encoded by a gene of interest and confer one or more functional characteristics upon the expression product. These amino acid sequences include sequences which will target the gene product for export from the cell (e.g., a secretory sequence) or to a subcellular compartment (e.g., the nucleus). Such amino acid sequences further include sequences which facilitate purification (e.g., a six His “tag”). Depending on the amino acid sequence and the function imparted by the fused sequence, the added amino acid sequences may or may not be cleaved from the translation product.

Fusion proteins also include proteins which have domains or regions derived from various different proteins. Examples of such a fusion protein are those containing domain II of Pseudomonas exotoxin, a domain or amino acid sequence which has binding affinity for a cell surface receptor associated with a particular cell type, and another amino acid sequence with a preselected biological activity. Domain II of Pseudomonas exotoxin will translocate across cell membranes. Using this system, fusion proteins can be designed which will bind to specific cells types, will translocate across the cytoplasmic membranes of these cells, and will catalyze predetermined intracellular biological reactions. A system of this type is described in Pastan et al., U.S. Pat. No. 5,705,163. Methods for identifying amino acid sequences, which bind to specific cell types are described in Wu, A., Nature Biotech. 14:429-431 (1996).

The nucleic acid molecules and expression systems of the invention may, in certain embodiments, further comprise genetic elements which confer additional functional characteristics such as selection markers.

Markers for the selection of prokaryotic and eukaryotic cells containing vectors the present invention are well known in the art and include puromycin, tetracycline, ampicillin, neomycin, hygromycin, gpt, zeocin, ouabain, blasticidin, bleomycin, and kanamycin resistance. Alternatively, selection markers such as DHRF, hisD, trpB, or glutamine synthetase may be used. Nucleotide sequences which result in high copy number amplification are also known in the art and include the ColE1 sequence contained in the pCytTs vector.

Recombinant Host Cells

The invention includes methods of making recombinant host cells and recombinant host cells produced using the methods of the invention. For example, the methods of the invention comprise introducing one or more nucleic acid molecules or expression systems described herein into a host cell. A variety of different recombinant host cells can be produced which contain the nucleic acid molecules and expression systems of the invention. Alphaviruses, for example, are known to have a wide host range. Sindbis virus, for example, infects cultured mammalian, reptilian, and amphibian cells, as well as some insect cells (Clark, H., J. Natl. Cancer Inst. 51:645 (1973); Leake, C., J. Gen. Virol. 35:335 (1977); Stollar, V. in THE TOGAVIRUSES, R. W. Schlesinger, Ed., Academic Press, (1980), pp. 583-621).

Thus, numerous host cells can be used in the practice of the invention. Representative host cells that may be used with the invention include, but are not limited to, bacterial cells, yeast cells, plant cells and animal cells. Preferred bacterial host cells include Escherichia spp. cells (particularly E. coli cells and most particularly E. coli strains DH10B, Stb12, DH5, DB3, DB3.1, DB4 and DB5), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Preferred animal host cells include insect cells (most particularly Drosophila melanogaster cells, Spodoptera frugiperda Sf9 and Sf21 cells and Trichoplusa High-Five cells), nematode cells particularly C. elegans cells), avian cells, amphibian cells (particularly Xenopus laevis cells), reptilian cells, and mammalian cells (most particularly NIH3T3, CHO, COS, VERO, BHK, HEK, other rodent cells, and human cells). Preferred yeast host cells include Saccharomyces cerevisiae cells and Pichia pastoris cells. BHK, COS, Vero, HeLa and CHO cells are particularly suitable for the production of heterologous polypeptides because they have the potential to glycosylate heterologous proteins in a manner similar to human cells (Watson, E. et al., Glycobiology 4:227, (1994)) and can be selected (Zang, M. et al., Bio/Technology 13:389 (1995)) or genetically engineered (Renner W. et al., Biotech. Bioeng. 47:476 (1995); Lee K. et al. Biotech. Bioeng. 50:336 (1996)) to grow in serum-free medium, as well as in suspension.

Example 2 shows that the expression system of the present invention can function in BHK cells and 293HEK cells. Moreover, we have previously demonstrated that the pCytTs system works efficiently in additional cells lines such as CHO-K1 and COS-7 (Boorsma et al., Nature Biotech. 18:429-432 (2000)).

The nucleic acid molecules and/or expression systems of the invention may be introduced into host cells using well known techniques of infection, transduction, electroporation, transfection, and transformation. Exemplary methods include DEAE-dextran mediated transfection, transient transfection, microinjection, cationic lipid-mediated transfection, scrape loading and ballistic introduction. Methods for the introduction of exogenous DNA sequences into host cells are discussed in Felgner, P. et al., U.S. Pat. No. 5,580,859. The nucleic acid molecules and/or vectors of the invention may be introduced alone or in conjunction with other nucleic acid molecules and/or vectors and/or proteins, peptides or RNAs. Alternatively, the nucleic acid molecules and/or expression systems of the invention may be introduced into host cells as a precipitate, such as a calcium phosphate precipitate, or in a complex with a lipid. Electroporation also may be used to introduce the nucleic acid molecules and/or expression systems of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into host cells are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Ausubel, F. et al., eds., CURRENT PROTOCOLS IN MOLECULAR BIOLOGY, John H. Wiley & Sons, Inc. (1997), Chapter 16), Watson, J. D., et al., Recombinant DNA, 2nd Ed., New York: W. H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

The invention is also directed to recombinant host cells comprising one or more nucleic acid molecules or expression systems of the invention. Also included within the present invention are in vitro cell cultures that comprise such recombinant host cells. Methods for producing recombinant host cells and cell cultures comprising the same are well-known in the art.

Production of Polypeptides and RNA Molecules

The nucleic acid molecules, expression systems and recombinant host cells of the invention may be used for the production of proteins, polypeptides and RNA molecules, e.g., untranslated RNA molecules. The methods of the invention may comprise, e.g., introducing one or more nucleic acid molecules or expression systems of the present invention into host cells to produce recombinant host cells, culturing the recombinant host cells under conditions suitable for expression of the polypeptide or untranslated RNA molecule, and recovering the polypeptide or untranslated RNA molecule.

The invention also provides methods for regulating the expression of a polypeptide or an untranslated RNA molecule. The methods of the invention may comprise, e.g., (a) introducing one or more nucleic acid molecules or expression systems of the invention into a host cell to produce a recombinant host cell, wherein said nucleic acid molecules or expression systems comprise a first open reading frame having a nucleotide sequence encoding a temperature-sensitive RNA-dependent RNA polymerase, (b) growing said recombinant host cell under suitable culture conditions, and (c) changing the temperature of the recombinant host cell culture from: (i) a permissive temperature to a restrictive temperature, or (ii) a restrictive temperature to a permissive temperature.

The present invention also provides methods for producing polypeptides and RNA molecules, said methods comprising introducing one or more nucleic acid molecules or expression systems of the invention into recombinant host cells, expanding the cells at a non-permissive temperature and incubating these cells at a permissive temperature. In a related aspect, the invention provides purified polypeptides and RNA molecules produced according to the methods of the present invention.

Depending on the molecule, which is expressed, it may be obtained either from the culture supematant or by lysing the recombinant host cells.

Polypeptides produced using the nucleic acid molecules and expression systems of the invention can be recovered and purified from recombinant cell cultures by methods known in the art including ammonium sulfate precipitation, anion or cation exchange chromatography, phosphocellulose chromatography, hydrophobic interaction chromatography, affinity chromatography, hydroxylapatite chromatography, and high performance liquid chromatography. Methods for purifying proteins are described in numerous sources (see, e.g., Celis, J., ed., CELL BIOLOGY, Academic Press, 2^{nd} edition, (1998)).

Untranslated RNA molecules produced using the nucleic acid molecules and expression systems of the invention can be recovered and purified from recombinant cell cultures by methods known in the art (see, e.g., Celis, J., ed., CELL BIOLOGY, Academic Press, 2^{nd} edition, (1998)). Methods for recovering and/or purifying RNA molecules include phenol/chloroform extraction, digestion with DNAses followed by precipitation of the undigested RNA molecules, and column chromatography (e.g., oligo dT column chromatography). Further, RNA molecules can be separated from other cellular material using the single-step guanidinium-thiocyanate-phenol-chloroform method described in Chomczynski and Sacchi, Anal. Biochem. 162:156-159 (1987).

The overall cell culture process employing nucleic acid molecules and expression systems of the invention for the production of polypeptides and/or untranslated RNA molecules can be implemented in a variety of bioreactor configurations (e.g., stirred-tank, perfused, membrane enclosed, encapsulated cell, fluidized bed, and air-lift reactors) and scales (from laboratory T-flasks to thousands of liters), chosen to accommodate the requirements of the host cell line utilized (e.g., anchorage dependency, O₂ concentrations), to maximize the production of expression product, and to facilitate subsequent recovery and purification of expression product.

The invention is also directed to the production of polypeptides or RNA molecules of interest using mammalian cells grown in serum-free or protein-free culture media. For example, by long-term culture under conditions restricting serum access or selecting for suspension growth, CHO cell lines are selected which are able to grow in serum-free medium and/or in suspension (Zang. M. et al., Bio/Technology 13:389 (1995)).

Further, a number of different bioprocess parameters can be varied in order to alter the glycosylation pattern of polypeptide products produced by the recombinant host cells of the invention. These factors include medium composition, pH, oxygen concentration, lack or presence of agitation, and, for the case of anchorage-dependent cells, the surface provided. Thus, the glycosylation pattern of glycoproteins may be altered by choosing the host cell in which these proteins are expressed in and the conditions under which the recombinant host cells are grown.

Pharmaceutical Compositions

The invention further provides pharmaceutical compositions comprising nucleic acid molecules and/or expression systems and/or recombinant host cells of the invention. The pharmaceutical compositions of the invention may comprise nucleic acid molecules and/or expression systems and/or recombinant host cells of the invention in combination (e.g., in solution) with a physiologically acceptable carrier and in a therapeutically effective amount. The administration of these pharmaceutical compositions may, for example, result in expression of a polypeptide in tissues of an animal which is immunogenic and intended to function as a vaccination. Similarly, the nucleic acid molecules and/or expression systems and/or recombinant host cells of the invention may carry sequences that encode polypeptides or RNA molecules required for the treatment of an active affliction. The administration of a pharmaceutical composition of the invention will thus be intended to have a therapeutic effect in these instances.

The nucleic acid molecules and/or expression systems and/or recombinant host cells of the invention will normally be administered to an individual in a pharmacologically acceptable carrier. A composition is said to be “pharmacologically acceptable” if its administration can be tolerated by a recipient individual. Further, the composition of the invention will be administered in a “therapeutically effective amount” (e.g., an amount that produces a desired physiological effect).

As would be understood by one of ordinary skill in the art, when the nucleic acid molecules and/or expression systems and/or recombinant host cells of the invention are administered to an individual, they may be in a composition which contains salts, buffers, adjuvants, or other substances which are desirable for improving the efficacy of the composition. Examples of materials suitable for use in preparing pharmaceutical compositions are provided in numerous sources including REMINGTON'S PHARMACEUTICAL SCIENCES (Osol, A, ed., Mack Publishing Co., (1980)).

The pharmaceutical compositions of the present invention can be administered by various art known means but will normally be administered by injection, infusion or other suitable physical methods. The compositions may alternatively be administered intramuscularly, intravenously, or subcutaneously. Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

When recombinant host cells are administered to an individual, the number of cells or nucleic acid molecules required to provide a therapeutically effective amount will vary with such factors as the individual's condition, the polypeptides or RNA molecules intended to be expressed, and the size of the individual.

Kits

The invention also provides kits comprising the isolated nucleic acid molecules, expression systems and/or recombinant host cells of the invention. The kits of the invention may optionally comprise one or more additional components selected from the group consisting of one or more containers (e.g., boxes, vials, tubes, jars ampules, etc.) one or more vectors, one or more nucleotides, one or more primers, one or more polypeptides having polymerase activity, one or more host cells (e.g., host cells that may be competent for uptake of nucleic acid molecules), and one or more buffers.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### Construction of the pCytTs Vector System

Manipulations and sequencing of DNA were carried out by standard procedures. The mutations in nsP2 were introduced by PCR using the following oligonucleotides:

oligo-nsp2 1:

(SEQ ID NO:3)

5′-AACATTGAAATCGATATTACAGGGG,
oligo-nsp2 2:

(SEQ ID NO:4)

5′-CGGGTTATGGTCGACCGGGC,
oligo-nsp2 3:

(SEQ ID NO:5)

5′-GTGCCCTCCCCTGAGTTTAAACAATTCAGGGCCGAACGCG,
and
oligo-nsp2 4:

(SEQ ID NO:6)

5′-GAATTGTTTAAACTCAGGAGGCACCCTCGTGG.

The single restriction sites used for first analysis and subsequent cloning (DraI, Clal and SalI) are underlined. PCR reactions were performed using either oligo-nsp2 1 (SEQ ID NO:3) and oligo-nsp2 3 (SEQ ID NO:5) or oligo-nsp2 2 (SEQ ID NO:4) and oligo-nsp2 4 (SEQ ID NO:6). 100 pmol of each oligo was used and 5 ng of the template DNA (pSinRep5; Xiong, C. etal, Science 243:1188-1191 (1989)) was used in the 100 Φ1 reaction mixture, containing 4 units of Taq or Pwo polymerase, 0.1 mM dNTPs and 1.5 MM MgSO₄. All DNA concentrations were determined photometrically using the GeneQuant apparatus (Pharmacia Biotech Inc., 800 Centennial Ave., Piscataway, N.J. 08854). The polymerase was added directly before starting the PCR reaction (starting point was 95° C.). The temperature cycles were as follows: 95° C. for 2 minutes, followed by 5 cycles of 95° C. (45 seconds), 58° C. (30 seconds), 72° C. (90 seconds) and followed by 25 cycles of 95° C. (45 seconds), 68° C. (30 seconds), 72° C. (90 seconds).

The two PCR fragment were purified using Qia spin PCR kit (QIAGEN, Inc., 9259 Eton Avenue, Chatsworth, Calif., 91311) and finally digested in an appropriate buffer using 20 units of SalI and DraI, respectively 20 units of ClaI and DraI. The digestion was performed for 12 hours at 37° C. The DNA fragments were gel-purified (Gene-Clean; Bio 101 Inc., 1070 Joshua Way, Vista, Calif., 92083, USA) and finally ligated into ClaI/SalI digested and gel-purified SinRep5 vector (Xiong, C. et al., Science 243:1188-1191 (1989). The correct sequence of the obtained vector was checked by DNA sequencing of the whole nsP2 gene.

The mutations in nsP4 were also introduced by PCR using the following oligonucleotides:

oligo-nsp4 1:

(SEQ ID NO:7)

5′-GGTAGACGAGACAGTCGCATGCCTGGATAC,
oligo-nsp4 2:

(SEQ ID NO:8)

5′-GTATCCAGGCATGCGACTGTCTCGTCTACC,
oligo-nsp4 3:

(SEQ ID NO:9)

5′-CAGACCGGTTAACGCCATAGCG TCG,
and
oligo-nsp4 4:

(SEQ ID NO:10)

5′-CTCTATTACTAGTATGGACAGTTGG,

The singular restriction sites used for the first analysis and the final cloning step (SphI, HpaI and SpeI) are underlined. Two PCR reactions were carried out as described above using either oligo-nsp4 1 (SEQ ID NO:7) and oligo-nsp4 3 (SEQ ID NO:9) or oligo-nsp4 2 (SEQ ID NO:8) and oligo-nsp4 4 (SEQ ID NO:10).

Both PCR products were gel-purified and then used in assembly PCR to amplify the whole nsP4 gene. For the assembly PCR, 50 pmol of the outer primers (3 and 4) and about 10 ng of each PCR fragment was used. The reaction volume was 100 μl, containing 4 units of Taq or Pwo polymerase, 0.1 mM dNTPs and 1.5 mM MgSO4. The PCR conditions were as followed:

95° C. for 2 minutes, followed by 5 cycles of 92° C. (45 seconds), 58° C. (30 seconds), 72° C. (120 seconds) and followed by 25 cycles of 92° C. (45 seconds), 64° C. (30 seconds), 72° C. (120 seconds).

The obtained PCR fragment was purified as described above and the eluate was digested with 20 units of SpeI and HpaI in an appropriate buffer. The fragment was gel-purified and ligated into gel-purified SpeI/HpaI restricted SinRepS vector. The correct sequence of the obtained vector was checked by DNA sequencing.

Overnight digestion of SinRepS-nsP4mut and SinRep5-nsp2mut with SpeI/HpaI and gel purification of the nsp4 fragment and sinRep-nsp2mut vector. The nsp4mut fragment was ligated into the SinRep5-nsp2mut vector. The final step was cloning the nsp gene into the 987/SinRep5 vector (Bredenbeek, P. et al., J. Virol. 67:6439-6446 (1993)) using ClaI and HpaI as restriction endonucleases, the resulting vector was named pCytTs (SEQ ID NO: 1).

Vector pCytTS2.1 (SEQ ID NO:2,) contains a different multiple cloning site than vector pCytTS, the RSV promoter is replaced by the CMV promoter, an SV40 intron sequence is included in the replicon and a puromycin resistance marker under control of the SV40 promoter is integrated on the plasmid. These elements can be cloned into pCytTS (SEQ ID NO: 1) in the following way:

Oligonucleotides Cyt-Link-FOR 5′-CTAGATTAATTAACTCGAG GCGCGCCG-3′ (SEQ ID NO: 11) and Cyt-Link-Rev 5′-GGCCCGGCGCGCCTCGAGTTAATTAAT-3′ (SEQ ID NO:12) are hybridized and ligated into XbaI/Bsp120I digested vector pCytTS. This step introduces a different multiple cloning site into pCytTS (IntermediateI).

The CMV promoter is introduced by assembled PCR using the following oligonucleotides:

5′CMV:

(SEQ ID No:13)

5′-ATAAGAATGCCGGCGGATCCGGCCATTAGC-3′
3′SinCMV:

(SEQ ID No:14)

5′-CCGTCAATACGGTTCACTAAACGAGCTCTGCTTATATAGACC-3′
5′CMV Sin:

(SEQ ID No:15)

5′-GCTCGTTTAGTGAACCGTATTGACGGCGTAGTACACAC-3′
3′Sin:

(SEQ ID No:16)

5′-ACGTCGGCCTCAATTTCGCG-3′

The singular restriction site used for the final cloning step (NgoAIV) is underlined. Two PCR reactions are carried out as described above using either oligonucleotide 5′ CMV 1 (SEQ ID NO:13) and 3′SinCMV (SEQ ID NO:14) or 5′CMVSin (SEQ ID NO:15) and 3′Sin (SEQ ID NO:16). As template DNA vector pLNCX (Miller et al., 1989, Biotechniques, 7, 980-982) and vector pCytTS can be used, respectively. Both PCR products are gel-purified and then used in assembly PCR. For the assembly PCR the outer primers (5′CMV and 3′Sin) are used, resulting in an 3089 bp size PCR fragment. The obtained PCR fragment is purified as described above and the eluate is digested with NgoAIV and Eco47III in an appropriate buffer. The fragment is gel-purified and ligated into gel-purified NgaAIV/Eco47III restricted IntermediateI. This cloning step replaces the RSV promoter in pCytTS with the CMV promoter (IntermediateII).

The SV40 introri is amplified by PCR from vector pcDNA1.1amp (Invitrogen) using the following oligonucleotides:

SVIntron-FOR:

(SEQ ID NO:17)

5′-GCGCGCGGGCCCAGAGGATCTTTGTGA AGG-3′
SVIntron-REV:

(SEQ ID NO:18)

5′-GCGCGCGCGGCCGCTACATCAAATATTT TTCC-3′

The PCR conditions can be choosen as followed: 94° C. for 2 minutes, followed by 30 cycles of 94° C. (45 seconds), 52° C. (30 seconds), 72° C. (60 seconds) and followed by a 7 min elongation step at 72° C. The obtained PCR fragment is purified using the Qia spin PCR kit (QIAGEN, Inc., 9259 Eton Avenue, Chatsworth, Calif., 91311). Finally the purified PCR fragment is digested in the corresponding buffer with Bsp120I/NotI and ligated into Bsp120I digested IntermediateII. The orientation of the insert is controlled by restriction enzyme analysis (IntermediateIII). The SV40 intron is thus introduced behind the multiple cloning site.

To introduce the puromycin resistance marker, vector pPUR (Clontech Laboratories, Inc., Cat. No. 6156-1) is modified first. Vector pPUR is digested with XbaI and the lineraized vector is treated with Klenow polymerase in order to get blunt ends. Religation of the vector destroyes the XbaI site. The resulting vector serves as DNA template in the PCR amplification of the puromycin resistance marker. The following oligonucleotides are used:

5′SV40PUR:

(SEQ ID NO:19)

5′- ACGTACGCGTGCGGCCGCGTTAGGGTGTGGAAAGTCCCC-3′
3′SV40PUR:

(SEQ ID NO:20)

5′- ACGTACGCGTTGGACAAACCACAACTAG AATGC-3′

The singular restriction site used for the cloning step (MluI) is underlined. PCR conditions can be choosen as described before with an annealing temperature at 45° C. The resulting PCR fragment is purified as described above, digested with MluI and ligated into MluI digested IntermediateIII. The resulting vector pCytTs2.1 (SEQ ID NO:2,) contains the puromycin resistance marker under control of the SV40 promoter.

### EXAMPLE 2

### Combination of the Temperature Sensitive pCytTs System with the Self Replacing EBNA System

This system was generated in order to rapidly generate cell populations, which inducibly express a gene of interest. In this system, the tightly regulated pCytTs system is combined with the episomally replicating EBV system.

A. Construction of Various pCytTs Constructs Containing Either the EBNA Origin of Replication (OriP) Alone or in Conjunction with the Replication Initiation Factor EBNA1.

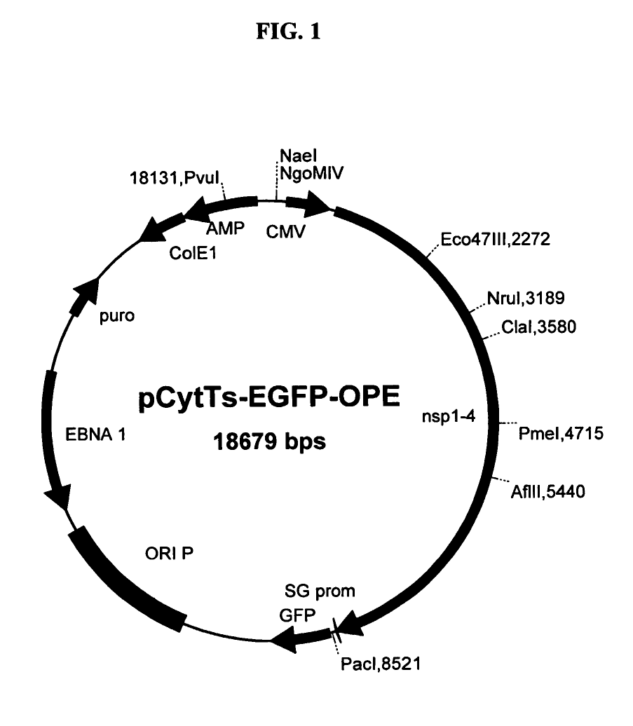

In order to be able to subclone either the origin of replication of Epstein-Barr virus alone or together with the replication initiation factor EBNA1 we first subcloned this two cassettes into a shuttle vector from which they can easily be transferred into our pCytTs system with or without various inserts. Combinatorial constructs with pCytTs mentioned in the Examples such as pCytTs-OriP, pCytTs-SEAP-OPE, pCytTs-SEAP or the like generally contain a CMV promoter. The sequence of the pCytTs with the CMV promoter is given in SEQ ID NO: 2 (pCytTs2.1; see Example 1). For sake of simplicity, however, the mentioned combinatorial constructs are abbreviated by simply stating pCytTs. The Epstein-Barr virus origin of replication OriP was amplified by PCR from pCEP4 (Invitrogen) using primers PH 51 and PH 52. The forward primer (PH 51) corresponds to nucleotides 261-280 of pCep4 and the reverse primer (PH 52) to nucleotides 2221-2239 of pCep4. PCR was performed using pfx polymerase (BRL) according to manufacturer's recommendations. The resulting PCR product (nucleotides 261-2239 of pCep4) was then cloned into pGEMTeasy vector (Invitrogen), which had previously been digested with Eco RI and treated with Klenow polymerase in order to get blunt ends. The resulting plasmid was termed pGemT-OriP. In order to get a subclonable cassette containing OriP and EBNA1 gene, pCep4 was digested with Dra III and Cla I. The resulting 4651 bp fragment was purified and blunt ends were generated by T4 DNA polymerase treatment. This fragment was then subcloned into pGEMTeasy vector (Invitrogen), which had previously been digested with Eco RI and treated with Klenow polymerase in order to get blunt ends. The resulting construct was termed pGemT-OPE. From these plasmids either the origin of replication alone (pGemT-OriP) or together with the replication initiation factor (pGemT-OPE) could be excised by Not I digestion and transferred into Not I sites of the various CytTs vectors (e.g. pCytTs vectors containing the RSV or CMV promoter (SEQ ID NO: 1 or SEQ ID NO: 2) which resulted in the vectors pCytTs-OriP (SEQ ID NO: 21), and pCytTs-OPE (SEQ ID NO: 22). pCytTs-IFNβ, pCytTs-EGFP, pCytTs-SEAP and pCytTs-cEPO vectors were linearized with Not I and the Not I fragment of pGemT-OPE was introduced by ligation leading to following constructs pCytTs-IFNβ-OPE pCytTs-EGFP-OPE (FIG. 1), pCytTs-SEAP-OPE and pCytTs-EPO-OPE. Similarly pCytTs-EPO-OriP and pCytTs-EGFP-OriP were generated by cloning the Not I fragment from pGemT-OriP into the Not I site of pCytTs-cEPO and pCytTs-EGFP respectively.

Initial experiments were performed with a construct in which a slightly truncated form of OriP had been subcloned into pCytTs-EGFP. pCep4 (Invitrogen) was digested with Dra III and Nsi I and the 1923 nucleotide fragment (position 200-2124 from pCep4) was then treated with T4 polymerase in order to get blunt ends. The blunted fragment was then subcloned into pCytTs-EGFP, which had previously been digested with Not I and blunted with T4 DNA polymerase. The resulting plasmid was termed pCytTs-EGFP-OriPd.

B. Analysis of pCytTs-EGFP-OriPd in 293 EBNA Cells

We first analyzed whether the introduction of Epstein-Barr virus derived origin of replication (OriP) into the pCytTs system would improve the inducible expression of a gene of interest on selected cell populations. We therefore transfected 293-EBNA cells (Invitrogen) with pCytTs-EGFP-OriPd. These cells constitutively express Epstein-Barr virus replication initiation factor EBNA1 and support OriP mediated DNA replication. 293-EBNA cells were transfected with pCytTs-EGFP-OriPd using Lipofectamine 2000 (BRL) according to the manufacturer's recommendation. 36 h after transfection cells were split 1 to 3 in presence of 1 μg/ml puromycin. After 1 to 2 days all non-transfected cells had detached and the resistant cells were further expanded under puromycin selection for another week. In order to asses the expression from the new vector, resistant cell populations were passed to two plates of which one was kept at 37° C. (uninduced) and one was shifted to 29° C. for induction of EGFP expression. 24 h after induction cells were harvested by trypsinisation and analysed by flow cytometry. The results are shown in Table 1. Upon induction cell populations carrying pCytTs-EGFP-OriPd were to 80% EGFP positive and displayed strong EGFP expression. In contrast cells, which had not been induced showed only weak EGFP expression and only about 8% of the cells, were green. Considering that stable cell populations containing CytTs-EGFP would only yield about 2-5% EGFP positive cells upon induction (data not shown) these results suggest that the OriP, which was introduced in the new vector, is functional and that it significantly improves the number of cells in a cell population, which expresses the gene of interest.

TABLE 1
GFP expression with pCytTS-EGFP-OriPd
293-EBNA cells 1 day after induction.

% GFP positive cells

| | 37° C. | 29° C. |
|---|---|---|
| pCytTS-EGFP-Orip | 8.2% | 79% |

C. Comparison of pCytTs-EGFP, pCytTs-EGFP-OriP and pCytTs-EGFP-OPE in BHK Cells

The following constructs, pCytTs-EGFP, pCytTs-EGFP-OriP and pCytTs-EGFP-OPE, were next examined in BHK (Baby hamster kidney) cells which are known to show tight regulation for the CytTs system. BHK cells were transfected with CytTs-EGFP, CytTs-EGFP-OriP and CytTs-EGFP-OPE using Lipofectamine 2000 (BRL) according to the manufacturer's recommendation. 24 h after transfection cells were split 1 to 3 in presence of 5 μg/ml puromycin. After 2 to 3 days all non-transfected cells had detached and the resistant cells were further expanded under puromycin selection for another week. Resistant cell populations were passed to two plates of which one was kept at 37° C. (non-induced) and one was shifted to 29° C. for induction of replicon replication and EGFP expression. Three days after induction cells were harvested and analysed by flow cytometry. The results are shown in Table 2. About 20% of the cell populations carrying CytTs-EGFP or CytTs-EGFP-OriP were EGFP positive upon induction, whereas four times more cells—up to 90% of the cells transfected with CytTs-EGFP-OPE did express EGFP after temperature induction. Thus, the inventive expression systems turned out to be very tight since less than 0.5% of the uninduced population showed weak EGFP expression.

TABLE 2
GFP expression with pCytTS-EGFP, pCytTS-EGFP-OriP and
pCytTS-EGFP- in BHK cells after 3 days of induction.

% GFP positive cells

| | 37° C. | 29° C. |
|---|---|---|
| pCytTS-EGFP | 0.20 | 19 |
| pCytTS-EGFP-OriP | 0.00 | 21 |
| pCytTS-EGFP-OPE | 0.00 | 93 |

D. Inducible Expression of pCytTs-IFNβ-OPE and pCytTs-SEAP-OPE in BHK Cells

We further analyzed constructs which contain both OriP and the replication initiation factor EBNA1 (pCytTs-IFNβ-OPE) with other genes of interest in BHK cells. These constructs do not depend on cell lines which have an integrated copy of the EBNA1 gene. As a gene of interest we choose IFNβ, which had already been expressed with the conventional CytTs system. To test whether our new vector system with its episomal replication could result in high, inducible levels of expression on selected cell populations we transfected BHK cells with pCytTs-IFNβ-OPE (see Example 2A) using Lipofectamine 2000 (BRL) according to the manufacturer's recommendation. 36 h after transfection cells were split 1 to 3 in the presence of 5 μg/ml puromycin. After 1 to 2 days all non-transfected cells had detached and the resistant cells were further expanded under puromycin selection for another 1-2 weeks. In order to assess the expression from the new vector, resistant cell populations were passed to two plates one of which was kept at 37° C. (uninduced) and one was shifted to 29° C. for induction of IFNβ expression. Tissue culture supernatants were harvested from the uninduced control plate and 3, 4, 7 and 9 day after induction from the plate that had been shifted to 29° C. The IFNβ production at the different time points was than quantitated by ELISA (Kit of Fujirebio Inc., Tokyo, Japan). The induced cell populations reached a maximal level of 1,000,000 IU/ml after 7-9 days of induction and no IFN production could be detected in cells kept at non-permissive temperature Table 3. These results show that our new vector system combining the CytTs system with the episomaly replicating EBNA system leads to high production levels in BHK cells on selected populations. The expression levels are at least as high as the ones obtained from a stable cell clone, which resulted from 4 subcloning rounds (data not shown). In addition the new system is still tightly regulated as demonstrated by up to 300,000 fold induction observed upon shifting the cells to 29° C.

TABLE 3
IFN-β production with pCytTS-IFN-OPE in
BHK cells after different days of induction.

| Days | IU/ml | Fold induction |
|---|---|---|
| uninduced | 3 | |
| 3 | 544410 | 159651 |
| 4 | 717990 | 210554 |
| 7 | 988880 | 289994 |
| 9 | 994140 | 291537 |

Besides IFNβ productin we also analysed SEAP production with our new vector system in BHK cells. Selected cell populations were generated as described under C using construct pCytTs-SEAP-OPE. After two weeks of selection and propagation the resistant cells were passed into two plates of which one was kept at 37° C. and the other was shifted to 29° C. for induction. Samples were taken from the uninduced cell populations and from the induced cell population 3, 4, 7 and 9 days after inductin. SEAP concentrations were measured in these samples using a kinetic ELISA for SEAP activity. The results are shown in Table 4.

TABLE 4
SEAP production with pCytTS-SEAP-OPE
after different days of induction

| Days | SEAP (mg/l) |
|---|---|
| uninduced | 0 |
| 3 | 22 |
| 4 | 66 |
| 7 | 125 |
| 9 | 268 |

Comparision of the EPO Production Derived from pCytTs-OriP-EPO or with pCytTs-OPE-EPO in EBNA Cells

We further investigated whether an additional copy of the EBNA-1 gene on the same plasmid leads to higher expression of the gene of interest in 293-EBNA cells. 293-EBNA cells were transfected either with pCytTs-OriP-EPO or with pCytTS-OPE-EPO (see Example 2A) using Lipofectamin 2000 (BRL) according to the manufacturer's recommendation. One day after transfection the cells were split 1 to 2.5 in the presence of 0.8 μg/ml puromycin. A stable cell pool was obtained by further keeping these cells under selective pressure for one week. Resistant cell populations were then passed to two plates. Once the cells had attached, one plate was kept at 37° C. and one plate was shifted to 29° C. for induction. Cell culture supernatants were collected from both plates 4 and 8 days after induction. The erythropoietin (EPO) levels in the cell culture supernatants were then determined with an ELISA kit (R&D Systems, Minneapolis, USA). As shown in Table 5 the additional copy of the EBNA1 gene on the plasmid leads to a 15 to 20 fold increase in the EPO production compared to the plasmid which only contains the OriP. In conclusion, this experiment shows that, even in cells which contain an integrated copy, the expression of the gene of interest can be increased by an additional copy on the plasmid. These results suggest that the EBNA1 dosage may be a limiting factor for efficient replication to occur. In these experiments the EBNA1 dosage was increased by a copy on the OriP-containing plasmid. Similar results may be obtained if the EBNA1 dosage is increased in the cells either by the integration of further copies or if the additional copies would be provided on a separate plasmid in trans.

TABLE 5
Improvement of expression with additional copies
of EBNA1 after 4 and 8 days of induction

EPO (mg/l)

| | pCytTS-EPO-OPE | pCytTS-EPO-OriP |
|---|---|---|
| uninduced | 0 | 0.02 |
| 4 | 2.7 | 0.2 |
| 8 | 3.9 | 0.2 |

### EXAMPLE 3

### Construction of Vectors of the pCytTs System Containing the Glutamine Synthetase as Selectable Marker

The glutamine synthetase gene is cloned either from hamster cells (CHO—K1, Chinese hamster ovary, ATCC, Cat. No. CCL-61; BHK21, Hamster Syrian kidney cells, DSMZ, Cat. No. ACC 61) or human cells (HEK 293 cells (ATCC, Cat. No. CRL-1573; Hela ATCC, Cat. No. CCL-2; Raji cells, ATCC, Cat. No. CCL-86; 293 EBNA , Invitrogen, Cat. No. R62007; and 143B cells, ATCC, Cat. No. CRL-8303) by the method of reverse transcription. Total RNA or cytoplasmic RNA is isolated from the cells using the RNeasy Kit (Qiagen, Inc., 9259 Eton Avenue, Chatsworth, Calif., 91311) according to the manufactures recommendation. Enrichment of poly(A)⁺ RNA from total RNA can be done by using the Oligotex mRNA Kit (Qiagen, Inc., 9259 Eton Avenue, Chatsworth, Calif., 91311). The resulting RNA can be treated with DNaseI to remove residual traces of DNA. cDNA synthesis is performed in the first step using the ThermoScript RT-PCR System (Invitrogen, Cat. No. 11146-024) with either total RNA or poly(A)+-selected RNA primed with oligo(dT) or random primers according to the manufactures recommendation. Treatment of cDNA with RNase H to remove the complementary RNA prior to PCR is optional. In the second step, PCR is performed using primers specific for the hamster or human glutamine synthetase. To amplify the cDNA of hamster and human glutamine synthetase the following oligonucleotides can be used:

hamGSfor:

(SEQ ID NO:23)

5′- GTCATGAAGCTTGCCACCGCTCAGAGC ACCTTC-3′
ham GSrev:

(SEQ ID NO:24)

5′- GACTTCTAGACTGGGGCGGGGTGGGATGAAC-3′
hGSfor:

(SEQ ID NO:25)

5′- GTCATGAAGCTTCTCGGCGACCAGAACACCTTC-3′
hGSrev:

(SEQ ID NO:26)

5′- GACTTCTAGACTGGGGCGGGGTGGGATGAAC

Restriction sites used for cloning are underlined (HindIII, XbaI). The resulting PCR fragment is purified using Qia spin PCR kit (Qiagen, Inc., 9259 Eton Avenue, Chatsworth, Calif., 91311).

To replace the puromycin resistance marker with the glutamine synthetase cassette in vector pCytTS2.1, the glutamine synthetase cassette is isolated from MluI and BamHI digested vector pGL3-GS. The fragment is treated with Klenow polymerase in order to get blunt ends and ligated into MluI digested vector pCytTS2. 1 treated with Klenow polymerase.

To combine the resulting pCytTS2.1gs vector with the self replicating EBNA system either the origin of replication alone (pGEMT-OriP) or together with the replication initiation factor (pGemT-OriP) can be excised by NotI digestion and transferred into the NotI site of pCytTS2.1gs.

Vectors of the present invention containing the glutamine synthetase gene as selection marker are transfected in different cell lines as described above (EXAMPLE 2). Mammalian cell lines that do not express GS (glutamine synthetase) cannot survive without added glutamine in the medium. For these cell lines, a transtected GS gene can function as a selectable marker by permitting growth in a glutamine-free medium (e.g. mouse myeloma NS0 cell line). For other cell lines (e.g. CHO—K1) producing endogenous GS, methionine sulphoximine (MSX) has to be added as selective agent to the glutamine-free medium.

### EXAMPLE 4

### Stability of Expression in Different Cell Lines Using the pCytTS-EBNA System

Vectors of the pCytTS-EBNA system containing either the EBNA origin of replication (OriP) alone or in conjunction with the replication initiation factor EBNA1 were transfected in different cell lines.

293 EBNA cells were transfected with vector pCytTS-EPO-OriP using Lipofectamine 2000 (BRL) according to the manufacturer's recommendation. One day after transfection cells were split 1 to 4 in the presence of 0.8 μg/ml puromycin. Several days after selection a stable cell pool was obtained which was further expanded. To asses inducible EPO expression, resistant cell populations were trypsinized and two T25 flask were seeded with three million cells each. Once the cells had attached one T25 flask was kept at 37° C. (uninduced) and one was shifted to 29° C. for induction of replicon replication and EPO expression. Cell culture supernatants were collected from both plates seven days after induction. The erythropoietin (EPO) level in the cell culture supernatants was determined with an ELISA kit (R&D Systems, Minneapolis, USA). A backup plate of the resistant cell pool was kept in culture and passaged about 1 to 10 twice a week. To determine stability of EPO expression over several weeks, cells from the backup plate were seeded at intervals of one or two weeks again on T25 flask as described above. Cells were induced for 7 days and EPO expression was determined by ELISA (Table 6).

TABLE 6
Stability of pCytTS-EPO-OriP in 293-EBNA cells between
1 and 5 weeks after completion of selection.

EPO (mg/l)

| Weeks | 37° C. | 29° C. |
|---|---|---|
| 1 | <0.1 | 2.3 |
| 2 | <0.1 | 2.8 |
| 3 | <0.1 | 4.4 |
| 4 | <0.1 | 4.9 |
| 5 | <0.1 | 4.5 |

These results show that stable cell pools containing a vector system combining the pCytTS system with the episomaly replicating EBNA system allow expression of EPO over several weeks without loss of productivity. EPO values in the range of 3-5 mg/l are obtained. Furtheron the pCytTS system is tightly regulated as at 37° C. hardly no EPO production can be detected.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. A nucleic acid molecule comprising:
(A) a first polynucleotide element which encodes an RNA molecule, said RNA molecule comprising:
(a) at least one cis-acting sequence element;
(b) a first nucleotide sequence comprising a first open reading frame, said first open reading frame having a nucleotide sequence encoding an RNA-dependent RNA polymerase; and
(c) at least one second nucleotide sequence selected from the group consisting of:
(i) a second open reading frame encoding a polypeptide;
(ii) a nucleotide sequence complementary to all or a part of the second open reading frame of (i); and
(iii) a nucleotide sequence encoding an untranslated RNA molecule or complement thereof;
wherein said second nucleotide sequence is operably linked to a promoter which is recognized by said RNA-dependent RNA polymerase;
(B) a second polynucleotide element comprising an origin of replication; and
(C) a third polynucleotide element encoding a replication initiation factor capable of recognizing said origin of replication.

2. The nucleic acid molecule of claim 1, wherein said origin of replication is derived from a prokaryotic organism, a eukaryotic organism or a virus.

3. The nucleic acid molecule of claim 2, wherein said origin of replication is derived from a eukaryotic organism selected from the group consisting of yeast, mammals and insects.

4. The nucleic acid molecule of claim 1, wherein said origin of replication is derived from a DNA virus that allows for episomal replication.

5. The nucleic acid molecule of claim 1, wherein said origin of replication is derived from a DNA virus, preferably from a DNA virus selected from the group consisting of Papillomavirus, Polyomavirus, Adenovirus, and Hepadnaviruses.

6. The nucleic acid molecule of claim 1, wherein said origin of replication is derived from a Herpesvirus, and preferably wherein said origin of replication is derived from Epstein-Barr virus (EBV).

7. The nucleic acid molecule of claim 1, wherein said origin of replication is oriP.

8. The nucleic acid molecule of claim 1, wherein said replication initiation factor is capable of operating as a plasmid maintenance factor.

9. The nucleic acid molecule of claim 1, wherein said replication initiation factor is derived from a prokaryotic organism, a eukaryotic organism or a virus.

10. The nucleic acid molecule of claim 1, wherein said replication initiation factor is derived from a DNA virus, preferably derived from a DNA virus selected from the group consisting of Herpesvirus, Papillomavirus, Polyomavirus, Adenovirus, and Hepadnavirus.

11. The nucleic acid molecule of claim 1, wherein said replication initiation factor is derived from a Herpesvirus, and preferably wherein said replication initiation factor is derived from EBV.

12. The nucleic acid molecule of claim 1, wherein said replication initiation factor is the EBNA-1 protein or a portion thereof.

13. The nucleic acid molecule of claim 1, wherein said replication initiation factor and said origin of replication are derived from the same organism or the same virus.

14. The nucleic acid molecule of claim 1, wherein said replication initiation factor and said origin of replication are derived from different organisms or viruses.

15. The nucleic acid molecule of claim 1 further comprising a fourth polynucleotide element, wherein said fourth polynucleotide element comprises a selection marker.

16. The nucleic acid molecule of claim 15, wherein said selection marker confers resistance to Puromycin.

17. The nucleic acid molecule of claim 1, wherein said second open reading frame is in a translatable format after one or more RNAdependent RNA replication events.

18. The nucleic acid molecule of claim 1, wherein said RNA-dependent RNA polymerase is selected from the group consisting of: (a) a temperature-sensitive RNA-dependent RNA polymerase; (b) a noncytopathic RNA-dependent RNA polymerase; and (c) a temperaturesensitive, non-cytopathic RNA-dependent RNA polymerase.

19. The nucleic acid molecule of claim 1, wherein said RNA-dependent RNA polymerase is of viral origin, and preferably wherein said RNA-dependent RNA polymerase is of alphaviral origin.

20. The nucleic acid molecule of claim 1, wherein said RNA-dependent RNA polymerase is derived from a Sindbis virus, a Semliki Forest virus or an Aura virus.

21. The nucleic acid molecule of claim 1, wherein said RNA-dependent RNA polymerase is derived from a virus selected from the group consisting of Bebaru virus, Cabassou virus, Chikungunya virus, Easter equine encephalomyelitis virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Venezuelan equine encephalomyelitis virus (VEE), and Ross River virus.

22. The nucleic acid molecule of claim 18, wherein said temperature-sensitive RNA-dependent RNA polymerase has replicase activity at temperatures below 34° C. which is at least five fold greater than the replicase activity exhibited at 34° C. or above.

23. The nucleic acid molecule of claim 18, wherein said temperature-sensitive RNA-dependent RNA polymerase has replicase activity at 34° C. which is at least five fold lower than the replicase activity exhibited at 29° C.

24. The nucleic acid molecule of any claim 1, wherein said second open reading frame encodes a cytokine, a lymphokine, a tumor necrosis factor, an interferon, a toxic polypeptide, a prodrug or a converting enzyme.

25. The nucleic acid molecule of claim 1, wherein said second nucleotide sequence encodes an untranslated RNA molecule selected from the group consisting of an antisense RNA molecule, a tRNA molecule, a rRNA molecule, and a ribozyme.

26. An expression system comprising:
(A) a first polynucleotide element which encodes an RNA molecule, said RNA molecule comprising:
(a) at least one cis-acting sequence element;
(b) a first nucleotide sequence comprising a first open reading frame, said first open reading frame having a nucleotide sequence encoding an RNA-dependent RNA polymerase; and
(c) at least one second nucleotide sequence selected from the group consisting of:
(i) a second open reading frame encoding a polypeptide;
(ii) a nucleotide sequence complementary to all or a part of the second open reading frame of (i); and
(iii) a nucleotide sequence encoding an untranslated RNA molecule or complement thereof;
wherein said second nucleotide sequence is operably linked to promoter which is recognized by said RNA-dependent RNA olymerase;
(B) a second polynucleotide element comprising an origin of replication; and
(C) a third polynucleotide element encoding a replication initiation factor capable of recognizing said origin of replication.

27. The expression system of claim 26, wherein said origin of replication is derived from a prokaryotic organism, a eukaryotic organism or a virus.

28. The expression system of claim 27, wherein said origin of replication is derived from an eukaryotic organism selected from the group consisting of yeast, insects and mammals.

29. The expression system of claim 26, wherein said origin of replication is derived from a DNA virus that allows for episomal replication.

30. The expression system of claim 26, wherein said origin of replication is derived from a DNA virus, preferably from a DNA virus selected from the group consisting of Herpesvirus, Papillomavirus, Polyomavirus, Adenovirus, and Hepadnaviruses.

31. The expression system of claim 26, wherein said origin of replication is derived from a Herpesvirus, and preferably wherein said origin of replication is derived from Epstein-Barr virus (EBV).

32. The expression system of claim 26, wherein said origin of replication is oriP.

33. The expression system of claim 1, wherein said replication initiation factor is capable of operating as plasmid maintenance factor.

34. The expression system of claim 1, wherein said replication initiation factor is derived from a prokaryotic organism or a eukaryotic organism, or a virus.

35. The expression system of claim 1, wherein said replication initiation factor is derived from a DNA virus, preferably derived from a DNA virus selected from the group consisting of Herpesvirus, Papillomavirus, Polyomavirus, Adenovirus, and Hepadnavirus.

36. The expression system of claim 1, wherein said replication initiation factor is derived from a Herpesvirus, and preferably wherein said replication initiation factor is derived from EBV.

37. The expression system of claim 1, wherein said replication initiation factor is the EBNA-1 protein, or portion thereof.

38. The expression system of claim 1, wherein said replication initiation factor and said origin of replication are derived from the same organism or the same virus.

39. The expression system of claim 1, wherein said replication initiation factor and said origin of replication are derived from 10 different organisms.

40. The expression system of claim 1, further comprising a fourth polynucleotide element, wherein said fourth polynucleotide element comprises a selection marker.

41. The expression system of claim 40, wherein said selection marker confers resistance to Puromycin.

42. The expression system of claim 1, wherein said second open reading frame is in a translatable format after one or more RNA-dependent RNA replication events.

43. The expression system of claim 1, wherein said first, second and third polynucleotide elements are DNA elements.

44. The expression system of claim 1, wherein said first, second and third polynucleotide elements are each present on a separate nucleic acid molecule.

45. The expression system of claim 1, wherein said first, second and third polynucleotide elements are all present on a single nucleic acid molecule.

46. The expression system of claim 1, wherein said first and said second polynucleotide elements are present on a first nucleic acid molecule, and said third polynucleotide element is present on a second nucleic acid molecule.

47. The expression system of claim 1, wherein said second nucleic acid molecule further comprises a fourth polynucleotide element capable of promoting the replication of said second nucleic acid molecule.

48. The expression system of claim 1, wherein said second nucleic acid sequence further comprises an origin of replication.

49. The expression system of claim 1, wherein said RNA-dependent RNA polymerase is selected from the group consisting of:
(a) a temperature-sensitive RNA-dependent RNA polymerase; (b) a noncytopathic RNA-dependent RNA polymerase; and (c) a temperature sensitive, non-cytopathic RNA-dependent RNA polymerase.

50. The expression system of claim 1, wherein said RNA-dependent RNA polymerase is of viral origin, and preferably wherein said RNA-dependent RNA polymerase is of alphaviral origin.

51. The expression system of claim 1, wherein said RNA-dependent RNA polymerase is derived from a Sindbis virus, from a Semliki Forest virus or from an Aura virus.

52. The expression system of claim 1, wherein said RNA-dependent RNA polymerase is derived from a virus selected from the group consisting of Bebaru virus, Cabassou virus, Chikungunya virus, Easter equine encephalomyelitis virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Venezuelan equine encephalomyelitis virus (VEE), and Ross River virus.

53. The expression system of claim 49, wherein said temperature-sensitive RNA-dependent RNA polymerase has replicase activity at temperatures below 34° C. which is at least five fold greater than the replicase activity exhibited at 34° C. or above.

54. The expression system of claim 49, wherein said temperature-sensitive RNA-dependent RNA polymerase has replicase activity at 34° C which is at least five fold lower than the replicase activity exhibited at 29° C.

55. The expression system of claim 1, wherein said second open reading frame encodes a cytokine, a lymphokine, a tumor necrosis factor, an interferon, a toxic protein, a prodrug or a converting enzyme.

56. The expression system of claim 1, wherein said second nucleotide sequence encodes an untranslated RNA molecule selected from the group consisting of an antisense RNA molecule, a tRNA molecule, a rRNA molecule, and a ribozyme.

57. A method of making a recombinant host cell comprising introducing the nucleic acid molecule of claim 1 into a host cell.

58. The method of claim 57, wherein said RNA-dependent RNA polymerase is selected from the group consisting of: (a) a temperature-sensitive RNAdependent RNA polymerase; (b) a non-cytopathic RNA-dependent RNA polymerase; and (c) a temperature-sensitive, non-cytopathic RNA-dependent RNA polymerase.

59. The method of claim 57, wherein said nucleic acid molecule is introduced by way of transfection.

60. A method of making a recombinant host cell comprising introducing the expression system of claim 26 into a host cell.

61. The method of claim 60, wherein said RNA-dependent RNA polymerase is selected from the group consisting of: (a) a temperature-sensitive RNAdependent RNA polymerase; (b) a non-cytopathic RNA-dependent RNA polymerase; and (c) a temperature-sensitive, non-cytopathic RNA-dependent RNA polymerase.

62. The method of claim 60, wherein said expression system is introduced by way of transfection.

63. A recombinant host cell produced by the method of claim 57.

64. A recombinant host cell produced by the method of claim 60.

65. A recombinant host cell comprising:
(A) a first polynucleotide element which encodes an RNA molecule, said RNA molecule comprising:
(a) at least one cis-acting sequence element;
(b) a first nucleotide sequence comprising a first open reading frame, said first open reading frame having a nucleotide sequence encoding an RNA-dependent RNA polymerase; and
(c) at least one second nucleotide sequence selected from the group consisting of:
(i) a second open reading frame encoding a polypeptide;
(ii) a nucleotide sequence complementary to all or a part of the open reading frame of (i); and
(iii) a nucleotide sequence encoding an untranslated RNA molecule or complement thereof;
wherein said second nucleotide sequence is operably linked to a promoter which is recognized by said RNA-dependent RNA polymerase;
(B) a second polynucleotide element comprising an origin of replication; and
(C) a third polynucleotide element encoding a replication initiation factor capable of recognizing said origin of replication.

66. The recombinant host cell of claim 65, wherein said third polynucleotide is stably integrated into the genome of said host cell.

67. The recombinant host cell of claim 65, wherein said first and said second polynucleotide elements are present on a first nucleic acid molecule, and said third polynucleotide element is present on a second nucleic acid molecule.

68. The recombinant host cell of claim 65, wherein said first, second and third polynucleotide elements are provided on the same nucleic acid molecule.

69. The recombinant host cell of claim 65, wherein said host cell comprises more than one copy of said third polynucleotide.

70. The recombinant host cell of claim 69, wherein at least one copy of said third polynucleotide is stably integrated into the genome of said host cell.

71. The recombinant host cell of claim 1, wherein said second open reading frame is in a translatable format after one or more RNA-dependent RNA replication events.

72. The recombinant host cell of claim 1 wherein said first, second and third polynucleotide elements are DNA elements.

73. The recombinant host cell of claim 1, further comprising a fourth polynucleotide comprising a selection marker, wherein said selection marker confers resistance to Puromycin.

74. The recombinant host cell of any claim 1, wherein said second nucleic acid molecule further comprises a fifth polynucleotide element capable of promoting the replication of said second nucleic acid molecule.

75. The recombinant host cell of claim 1, wherein said nucleotide sequence further comprises an origin of replication.

76. The recombinant host cell of claim 1, wherein said RNA-dependent RNA polymerase is selected from the group consisting of: (a) a temperature-sensitive RNA-dependent RNA polymerase; (b) a noncytopathic RNA-dependent RNA polymerase; and (c) a temperaturesensitive, non-cytopathic RNA-dependent RNA polymerase.

77. The recombinant host cell of claim 1, wherein said recombinant host cell is a mammalian cell, and wherein preferably said mammalian cell is selected from the group consisting of a human cell, a primate cell and a rodent cell.

78. A method for producing a polypeptide or untranslated RNA molecule, said method comprising:
(a) introducing the nucleic acid molecule of claim 1 into a host cell to produce a recombinant host cell; and
(b) culturing said recombinant host cell under conditions suitable for expression of said polypeptide or untranslated RNA molecule.

79. The method of claim 78, further comprising recovering said polypeptide or untranslated RNA molecule.

80. A method for producing a polypeptide or untranslated RNA molecule, said method comprising:
(a) introducing the expression system of claim 26 into a host cell to produce a recombinant host cell; and
(b) culturing said recombinant host cell under conditions suitable for expression of said polypeptide or untranslated RNA molecule.

81. The method of claim 80, further comprising recovering said polypeptide or untranslated RNA molecule.

82. A method for regulating the expression of a polypeptide or an untranslated RNA molecule, said method comprising:
(a) introducing the nucleic acid molecule of claim 18 into a host cell to produce a recombinant host cell;
(b) growing said recombinant host cell under suitable culture conditions; and
(c) changing the temperature of the recombinant host cell culture from:
(i) a permissive temperature to a restrictive temperature, or
(ii) a restrictive temperature to a permissive temperature;
wherein said polypeptide or untranslated RNA molecule is encoded by said first polynucleotide.

83. The method of claim 82, wherein said polypeptide is a polypeptide that is toxic to said host cell.

84. A method for regulating the expression of a polypeptide or an untranslated RNA molecule, said method comprising:
(a) introducing the expression system of claim 49 into a host cell to produce a recombinant host cell;
(b) growing said recombinant host cell under suitable culture conditions; and
(c) changing the temperature of the recombinant host cell culture from:
(i) a permissive temperature to a restrictive temperature, or
(ii) a restrictive temperature to a permissive temperature;
wherein said polypeptide or untranslated RNA molecule is encoded by said first polynucleotide.

85. The method of claim 84, wherein said polypeptide is a polypeptide that is toxic to said host cell.


SEQUENCE LISTING (26 sequences)

SEQ ID NO: 1 - DNA, 11282 bp, Artificial Sequence
  cDNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcaattaaccctcactaaagggaacaaaagctggctagtggatccagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgccctacctcgataccgtcgagattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgtttaaactcaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggattcatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgagacagtcgcatgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccacctctagacgcgtagatctcacgtgagcatgcaggccttgggcccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagggaattcccaacttgtttattgcagcttataatggttacaaataaagcaatagcatcacaaatttcacaaataaagcatttttttcactgcattctagttgtggtttgtccaaactcatcaatgtatcttatcatgtctggatccgtcgagacgcgtccaattcgccctatagtgagtcgtattacgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 13068 bp, Artificial Sequence
  cDNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgtttaaactcaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggattcatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgagacagtcgcatgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaataccacaacaccaccacctctagattaattaactcgaggcgcgccgggcccagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtagcggcccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaggggaattcccaacttgtttattgcagcttataatggttacaaataaagcaatagcatcacaaatttcacaaataaagcatttttttcactgcattctagttgtggtttgtccaaactcatcaatgtatcttatcatgtctggatccgtcgagacgcgtgcggccgcgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccacccggggcggccccgccgaccccgcacccgcccccgaggcccaccgactctagctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaacgcgtcggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgtttcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 25 bp, Artificial Sequence
  primer
aacattgaaatcgatattacagggg

SEQ ID NO: 4 - DNA, 20 bp, Artificial Sequence
  primer
cgggttatggtcgaccgggc

SEQ ID NO: 5 - DNA, 40 bp, Artificial Sequence
  primer
gtgccctcccctgagtttaaacaattcagggccgaacgcg

SEQ ID NO: 6 - DNA, 32 bp, Artificial Sequence
  primer
gaattgtttaaactcaggaggcaccctcgtgg

SEQ ID NO: 7 - DNA, 30 bp, Artificial Sequence
  primer
ggtagacgagacagtcgcatgcctggatac

SEQ ID NO: 8 - DNA, 30 bp, Artificial Sequence
  primer
gtatccaggcatgcgactgtctcgtctacc

SEQ ID NO: 9 - DNA, 25 bp, Artificial Sequence
  primer
cagaccggttaacgccatagcgtcg

SEQ ID NO: 10 - DNA, 25 bp, Artificial Sequence
  primer
ctctattactagtatggacagttgg

SEQ ID NO: 11 - DNA, 27 bp, Artificial Sequence
  primer
ctagattaattaactcgaggcgcgccg

SEQ ID NO: 12 - DNA, 27 bp, Artificial Sequence
  primer
ggcccggcgcgcctcgagttaattaat

SEQ ID NO: 13 - DNA, 30 bp, Artificial Sequence
  primer
ataagaatgccggcggatccggccattagc

SEQ ID NO: 14 - DNA, 42 bp, Artificial Sequence
  primer
ccgtcaatacggttcactaaacgagctctgcttatatagacc

SEQ ID NO: 15 - DNA, 38 bp, Artificial Sequence
  primer
gctcgtttagtgaaccgtattgacggcgtagtacacac

SEQ ID NO: 16 - DNA, 20 bp, Artificial Sequence
  primer
acgtcggcctcaatttcgcg

SEQ ID NO: 17 - DNA, 30 bp, Artificial Sequence
  primer
gcgcgcgggcccagaggatctttgtgaagg

SEQ ID NO: 18 - DNA, 32 bp, Artificial Sequence
  primer
gcgcgcgcggccgctacatcaaatatttttcc

SEQ ID NO: 19 - DNA, 39 bp, Artificial Sequence
  primer
acgtacgcgtgcggccgcgttagggtgtggaaagtcccc

SEQ ID NO: 20 - DNA, 33 bp, Artificial Sequence
  primer
acgtacgcgttggacaaaccacaactagaatgc

SEQ ID NO: 21 - DNA, 15081 bp, Artificial Sequence
  cDNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgtttaaactcaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggattcatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgagacagtcgcatgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaataccacaacaccaccacctctagattaattaactcgaggcgcgccgggcccagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtagcggcccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaggggaattcccaacttgtttattgcagcttataatggttacaaataaagcaatagcatcacaaatttcacaaataaagcatttttttcactgcattctagttgtggtttgtccaaactcatcaatgtatcttatcatgtctggatccgtcgagacgcgtgcggccgcgggaattcgatctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgcatcactagtgaattcgcggccgcgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccacccggggcggccccgccgaccccgcacccgcccccgaggcccaccgactctagctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaacgcgtcggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgtttcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 22 - DNA, 17753 bp, Artificial Sequence
  cDNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtattgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgtttaaactcaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggattcatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgagacagtcgcatgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaataccacaacaccaccacctctagattaattaactcgaggcgcgccgggcccagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtagcggcccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaggggaattcccaacttgtttattgcagcttataatggttacaaataaagcaatagcatcacaaatttcacaaataaagcatttttttcactgcattctagttgtggtttgtccaaactcatcaatgtatcttatcatgtctggatccgtcgagacgcgtgcggccgcgggaattcgatgtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatatcactagtgaattcgcggccgcgttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccacccggggcggccccgccgaccccgcacccgcccccgaggcccaccgactctagctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaacgcgtcggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgtttcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 23 - DNA, 33 bp, Artificial Sequence
  primer
gtcatgaagcttgccaccgctcagagcaccttc

SEQ ID NO: 24 - DNA, 31 bp, Artificial Sequence
  primer
gacttctagactggggcggggtgggatgaac

SEQ ID NO: 25 - DNA, 33 bp, Artificial Sequence
  primer
gtcatgaagcttctcggcgaccagaacaccttc

SEQ ID NO: 26 - DNA, 31 bp, Artificial Sequence; identical to SEQ ID NO: 24
  primer